(19) United States
(12) Patent Application Publication
(10) Pub. No.: US 2013/0079364 A1
(43) Pub. Date: Mar. 28, 2013

(54) Peripheral Opioid Agonists and Peripheral Opioid Antagonists

(71) Applicants: Thomas E. Jenkins, Half Moon Bay, CA (US); Craig O. Husfeld, San Mateo, CA (US)

(72) Inventors: Thomas E. Jenkins, Half Moon Bay, CA (US); Craig O. Husfeld, San Mateo, CA (US)

(73) Assignee: SIGNATURE THERAPEUTICS, INC., San Carlos, CA (US)

(21) Appl. No.: 13/634,532

(22) PCT Filed: Apr. 8, 2011
(86) PCT No.: PCT/US11/31844

§ 371 Date: Nov. 16, 2012

Related U.S. Application Data
(60) Provisional application No. 61/326,617, filed on Apr. 21, 2010.

(51) Int. Cl. A61K 31/439; C07D 489/02

(52) U.S. Cl. CPC A61K 31/439; C07D 489/02
USPC 514/282; 546/46

(57) ABSTRACT

The present disclosure provides compositions, and their methods of use, where the compositions comprise a ketone-modified opioid drug, wherein the drug comprises a ketone-modified opioid and a substituent on the opioid that mediates retention of the drug in the peripheral nervous system as opposed to the central nervous system following ingestion by a subject.

### CROSS-REFERENCE TO RELATED APPLICATIONS

This application claims priority benefit of U.S. provisional application Ser. No. 61/326,617, filed Apr. 21, 2010, which application is incorporated herein by reference in its entirety.

### INTRODUCTION

Natural and synthetic alkaloids of opium (i.e., opioids) are useful as analgesics for the treatment of severe pain. Opioids target three types of endogenous opioid receptors: mu-, delta-, and kappa-receptors. Many opioids are mu-receptor agonists that are highly efficacious analgesic compounds due to their activation of opioid receptors in the brain and central nervous system (CNS). Opioid receptors are, however, not only limited to the CNS, but also may be found in other tissues throughout the body. These receptors located outside the CNS are referred to as peripheral opioid receptors.

Peripheral opioid agonists can activate peripheral opioid receptors to effect analgesia, such as, but not limited to, relief from inflammatory pain or neuropathic pain. However, unless the opioid agonists are peripherally-restricted, they can also lead to abuse, misuse, overdose or respiratory depression. Non-steroidal anti-inflammatory drugs (NSAIDs) are also analgesic but can lead to gastrointestinal or cardiovascular side effects.

In addition, opioid agonists cause side effects when they interact with peripheral opioid receptors, for example, in the gastrointestinal tract. These side effects can be countered by co-administering a peripheral opioid antagonist, such as N-methylnaltrexone. The selective action of these antagonists for peripheral opioid receptors arises from their poor ability to cross the blood brain barrier. Such peripheral opioid antagonists, however, are also poorly absorbed through the gastrointestinal tract, and therefore need to be administered by injection. There is a need for a peripheral opioid antagonist that exhibits strong receptor potency, that is peripherally-restricted and that is orally bioavailable (i.e., is absorbable through the gastrointestinal tract when administered orally. There is also a need for a peripheral opioid agonist that has these features. Such peripheral opioid antagonists and peripheral opioid agonists could be administered orally and would be safer than their non-peripherally-restricted counterparts.

### SUMMARY

The present disclosure provides compositions, and their methods of use, where the compositions comprise a ketone-modified opioid drug, wherein the drug comprises a ketone-modified opioid and a substituent on the opioid that mediates retention of the drug in the peripheral nervous system as opposed to the central nervous system following ingestion by a subject. Such ketone-modified opioid drugs exhibit receptor potency, are peripherally restricted, and can be administered orally, in view of their absorbability through the gastrointestinal tract.

The present disclosure provides a compound of formula (I):

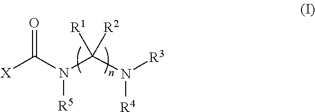

wherein:

X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;

R⁵ is selected from hydrogen, alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 10;

R³ is selected from hydrogen, alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or R⁴ is

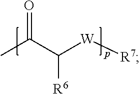

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and

provided that:

1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is

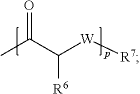

or a salt, hydrate or solvate thereof.

The present disclosure provides a compound a compound of formula (V):

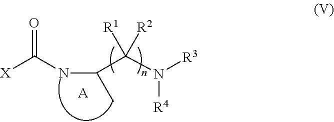

wherein:

X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;

the A ring is a heterocyclic 5 to 12-membered ring;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

n is an integer from 1 to 10;

R³ is selected from hydrogen, alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or R⁴ is

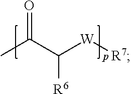

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and

provided that:

1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is

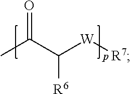

or a salt, hydrate or solvate thereof.

### BRIEF DESCRIPTION OF THE FIGURES

FIG. 1 compares mean plasma concentrations over time of Compound 1 and of naltrexone released from Compound 1 upon oral administration of Compound 1 to rats.

FIG. 2 compares mean plasma concentration over time of Compound 1 and of naltrexone released from Compound 1 upon intravenous administration of Compound 1 to rats.

FIG. 3 compares mean plasma concentrations over time of Compound 2 and of oxycodone released from Compound 2 upon oral administration of Compound 2 to rats.

FIG. 4 compares mean plasma concentration over time of Compound 2 and of oxycodone released from Compound 2 upon intravenous administration of Compound 2 to rats.

FIG. 5 compares mean plasma concentrations over time of Compound 3 and of naltrexone released from Compound 3 upon oral administration of Compound 3 to rats.

FIG. 6 compares mean plasma concentration over time of Compound 3 and of naltrexone released from Compound 3 upon intravenous administration of Compound 3 to rats.

FIG. 7 compares mean plasma concentrations over time of Compound AG-4 and of hydromorphone released from Compound AG-4 upon oral administration of Compound AG-4 to rats.

FIG. 8 compares the effects of subcutaneous administration to rats pre-treated with morphine of peripheral opioid antagonists Compound AN-1 and Compound AN-6 to that of naltrexone in a tail flick latency assay.

FIG. 9 compares GI transit efficacy in rats upon oral administration of hydromorphone without or with peripheral opioid antagonists of the embodiments.

FIG. 10 compares anti-inflammatory effects over time in an inflammatory paw model of rats administered peripheral opioid agonist Compound AG-2 or of untreated rats.

### TERMS

The following terms have the following meaning unless otherwise indicated. Any undefined terms have their art recognized meanings.

“Alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group is specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Carboxyl,” “carboxy” or “carboxylate” refers to —CO₂H or salts thereof.

“Cyano” or “nitrile” refers to the group —CN.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to —O—, —S—, —S—S—, —O—S—, —NR³⁷R³⁸—, .═N—N═, —N═N—, —N═N—NR³⁹R⁴⁰, —PR⁴¹—, P(O)₂—, —POR⁴²—, —O—P(O)₂, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These hetero atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶)(O⁻), —OP(O)(OR⁶)(OR⁶¹), C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S, —NR⁶⁰R⁶¹, ═NR⁶⁰, CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to substituted aryl-(substituted aryl)-substituted aryl.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

“Opioid” refers to a chemical substance that exerts its pharmacological action by interaction at opioid receptors. Opioids can be agonists, antagonists, or partial agonists, partial antagonists or partial agonists and antagonists. An “opioid agonist” is a compound that effects a positive response when it binds to an opioid receptor; for example, an opioid agonist can effect analgesia or sedation. An “opioid antagonist” is a compound that binds to the opioid receptor but does not activate the receptor to effect the response that an opioid agonist effects. An opioid antagonist can block the activity of an opioid agonist. A “peripheral opioid agonist” is a compound that is not capable of penetrating the blood brain bather or has a greatly reduced ability to cross the blood brain barrier and exerts its positive response (e.g., analgesia or sedation) by binding to opioid receptors outside the central nervous system. A “peripheral opioid antagonist” is a compound that is not capable of penetrating the blood brain barrier or has a greatly reduced ability to cross the blood brain bather and hence is capable of antagonizing the (undesired) action of an opioid agonist outside the central nervous system.

“Pharmaceutically acceptable carrier” refers to a diluent, adjuvant, excipient or vehicle with, or in which a compound is administered.

“Pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutical composition” refers to at least one compound and can further comprise a pharmaceutically acceptable carrier, with which the compound is administered to a patient.

The term “solvate” as used herein refers to a complex or aggregate formed by one or more molecules of a solute, e.g. a ketone-modified opioid drug or a pharmaceutically acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

“Preventing” or “prevention” or “prophylaxis” refers to a reduction in risk of occurrence of a condition, such as pain.

“Therapeutically effective amount” means the amount of a compound that, when administered to a patient for preventing or treating a condition such as pain, is sufficient to effect such treatment. The “therapeutically effective amount” will vary depending on the compound, the condition and its severity and the age, weight, etc., of the patient.

“Treating” or “treatment” of any condition, such as pain, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a compound refers to one or more compounds. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

The nomenclature used herein to name the subject compounds is illustrated in the Examples herein. In certain instances, this nomenclature has generally been derived using the commercially-available AutoNom software (MDL, San Leandro, Calif.).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

### Representative Embodiments

Reference will now be made in detail to various embodiments. It will be understood that the invention is not limited to these embodiments. To the contrary, it is intended to cover alternatives, modifications, and equivalents as may be included within the spirit and scope of the allowed claims.

The present disclosure provides compositions, and their methods of use, where the compositions comprise a ketone-modified opioid drug, wherein the drug comprises a ketone-modified opioid and a substituent on the opioid that mediates retention of the drug in the peripheral nervous system as opposed to the central nervous system following ingestion by a subject.

As used herein, a ketone-modified opioid drug refers to a ketone-modified opioid and a substituent on the opioid that mediates retention of the drug in the peripheral nervous system following ingestion by a subject. In certain embodiments, resistance to enzyme (e.g. trypsin) cleavage of the ketone-modified opioid drug aids in retention of the drug in the peripheral nervous system. A ketone-modified opioid drug of the embodiments does not substantially penetrate the blood brain bather and, surprisingly, is well absorbed through the gastrointestinal system when administered orally. In addition, such a ketone-modified opioid drug is stable and potent.

In a ketone-modified opioid drug, the substituent that mediates retention of the drug in the peripheral nervous system is attached to the ketone-containing opioid through the enolic oxygen atom of the ketone moiety such that the hydrogen atom of the corresponding enolic group of the ketone-containing opioid is replaced by a covalent bond to the substituent. The disclosure also provides for a ketone-modified opioid drug, where a substituent is attached to the ketone-containing opioid through the enolic oxygen atom of the ketone moiety such that the hydrogen atom of the corresponding enolic group of the ketone-containing opioid is replaced by a covalent bond to the substituent and the olefin of the corresponding enol is reduced. The disclosure also provides for a ketone-modified opioid drug, where a substituent is attached to the ketone-containing opioid through an amino group that is generated from reductive amination of the ketone of a ketone-containing opioid.

The substituent comprises at least one of the following structural features: an amino acid of D-configuration and/or alkylation or arylation of an amino group on the substituent. As used herein, a D-amino acid refers to an amino acid of D-configuration. In certain instances the structural feature is L-proline.

Examples of the opioid drugs and portions thereof are described below.

### Ketone-Containing Opioids

An “opioid” refers to a chemical substance that exerts its pharmacological action by interaction at an opioid receptor. The disclosure provides for opioid agonists and opioid antagonists. An opioid can be an isolated natural product, a synthetic compound or a semi-synthetic compound. “Ketone-containing opioid” refers to a subset of the opioids that contain a ketone group. As used herein, a ketone-containing opioid is an opioid containing an enolizable ketone group. A ketone-containing opioid is a compound with a pharmacophore that presents to the opioid receptor an aromatic group and an aliphatic amine group in an architecturally discrete way. See, for example, Foye's Principles of Medicinal Chemistry, Sixth Edition, ed. T. L. Lemke and D. A. Williams, Lippincott Williams & Wilkins, 2008, particularly Chapter 24, pages 653-678.

The disclosure provides for a ketone-modified opioid drug, wherein the opioid has an optionally substituted morphinan structure:

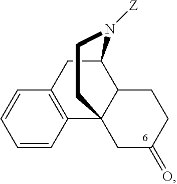

wherein the ketone is situated at the 6-position of the morphinan structure and Z is hydrogen or other group, such as, but not limited to, alkyl or substituted alkyl.

In certain embodiments, the opioid has the following optionally substituted morphinan structure:

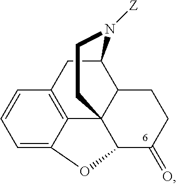

wherein the ketone is situated at the 6-position of the morphinan structure and Z is hydrogen or other group, such as, but not limited to, alkyl or substituted alkyl.

In certain embodiments, the opioid has the following optionally substituted morphinan structure:

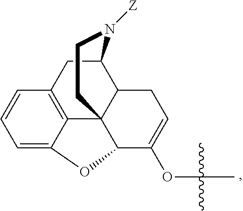

wherein Z is hydrogen or other group, such as, but not limited to, alkyl or substituted alkyl. The structure is shown as an enol, in which attachment to the substituent is through the enolic oxygen atom of the ketone moiety such that the hydrogen atom of the corresponding enolic group of the ketone-containing opioid is replaced by a covalent bond to the substituent.

In certain embodiments, the opioid has the following reduced enol structure:

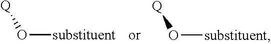

wherein Q is an opioid. The structure is shown as an enol with a reduced olefin, in which attachment to the substituent is through the enolic oxygen atom of the ketone moiety such that the hydrogen atom of the corresponding enolic group of the ketone-containing opioid is replaced by a covalent bond to the substituent.

In certain embodiments, the opioid has the following optionally substituted morphinan structure:

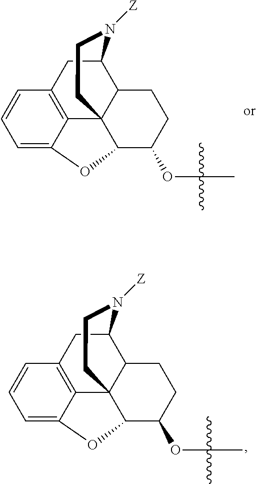

wherein Z is hydrogen or other group, such as, but not limited to, alkyl or substituted alkyl. The structure is shown as an enol with a reduced olefin, in which attachment to the substituent is through the enolic oxygen atom of the ketone moiety such that the hydrogen atom of the corresponding enolic group of the ketone-containing opioid is replaced by a covalent bond to the substituent.

In certain embodiments, the opioid has the following optionally substituted morphinan

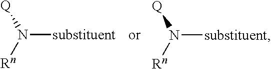

wherein Q is an opioid and Rⁿ is hydrogen or other group, such as, but not limited to, alkyl or substituted alkyl. The structure is shown as a ketone that has been reductively aminated, in which attachment to the substituent is through the corresponding amino group that is generated from reductive amination of the ketone moiety such that the hydrogen atom of the corresponding amino group is replaced by a covalent bond to the substituent.

In certain embodiments, the opioid has the following optionally substituted morphinan structure:

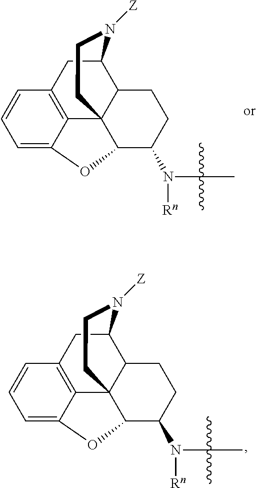

wherein Z is hydrogen or other group, such as, but not limited to, alkyl or substituted alkyl and Rⁿ is hydrogen or other group, such as, but not limited to, alkyl or substituted alkyl. The structure is shown as a ketone that has been reductively aminated, in which attachment to the substituent is through the corresponding amino group that is generated from reductive amination of the ketone moiety such that the hydrogen atom of the corresponding amino group is replaced by a covalent bond to the substituent.

For example, ketone-containing opioids include, but are not limited to, acetylmorphone, hydrocodone, hydromorphone, naloxone, N-methylnaloxone, naltrexone, N-methylnaltrexone, oxycodone, oxymorphone, and pentamorphone. Other examples include, but are not limited to, ketobemidone and methadone. The foregoing opioids may or may not include a morphinan core structure.

In certain embodiments, the ketone-containing opioid is hydrocodone or oxycodone. In certain embodiments, the ketone-containing opioid is hydromorphone or oxymorphone.

In certain embodiments, the ketone-containing opioid is naloxone or naltrexone. In certain embodiments, the ketone-containing opioid is N-methylnaloxone or N-methylnaltrexone.

It is contemplated that opioids bearing at least some of the functionalities described herein will be developed; such opioids are included as part of the scope of this disclosure.

### Ketone-Modified Opioid Drugs

The disclosure provides for ketone-modified opioid drugs. Such drugs can be ketone-modified opioid agonist drugs or ketone-modified opioid antagonist drugs, and, as such, include ketone-modified opioid partial agonist and/or partial antagonist drugs.

The disclosure provides for compounds of formulae (I)-(IV).

Formula (I)

In one of its composition aspects, the present embodiments provide a compound of formula (I):

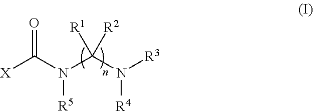

wherein:

X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;

R⁵ is selected from hydrogen, alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 10;

R³ is selected from hydrogen, alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or R⁴ is

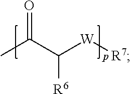

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and

provided that:

1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is

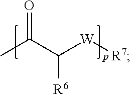

or a salt, hydrate or solvate thereof.

Formula (II)

In one of its composition aspects, the present embodiments provide a compound of formula (II):

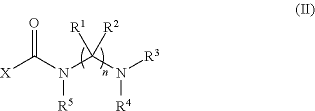

wherein:

X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;

R⁵ is selected from hydrogen, alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 10;

R³ is selected from alkyl, substituted alkyl, aryl, and substituted aryl;

R⁴ is

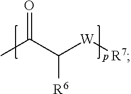

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol;

or a salt, hydrate or solvate thereof.

Formula (III)

In one of its composition aspects, the present embodiments provide a compound of formula (III):

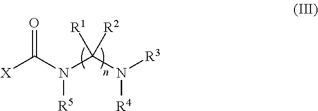

wherein:

X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;

R⁵ is selected from hydrogen, alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 10;

R³ is selected from alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or a salt, hydrate or solvate thereof.

Formula (IV)

In one of its composition aspects, the present embodiments provide a compound of formula (IV):

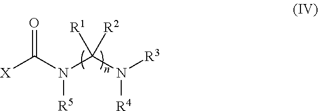

wherein:

X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;

R⁵ is selected from hydrogen, alkyl, substituted alkyl, arylalkyl, substituted arylalkyl, aryl and substituted aryl;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;

n is an integer from 2 to 10;

R³ is hydrogen;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or a salt, hydrate or solvate thereof.

The disclosure provides for compounds of formulae (V)-(VIII).

Formula (V)

In one of its composition aspects, the present embodiments provide a compound of formula (V):

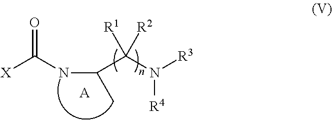

wherein:

X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;

the A ring is a heterocyclic 5 to 12-membered ring;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

n is an integer from 1 to 10;

R³ is selected from hydrogen, alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or R⁴ is

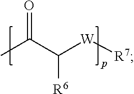

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and

provided that:

1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is

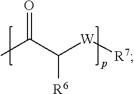

or a salt, hydrate or solvate thereof.

Formula (VI)

In one of its composition aspects, the present embodiments provide a compound of formula (VI):

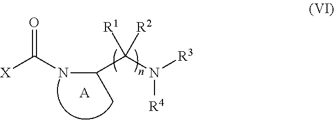

wherein:

X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;

the A ring is a heterocyclic 5 to 12-membered ring;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

n is an integer from 1 to 10;

R³ is selected from alkyl, substituted alkyl, aryl, and substituted aryl;

R⁴ is

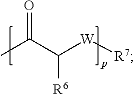

each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;

each W is independently —NR⁸—;

each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;

p is an integer from one to five; and

R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol;

or a salt, hydrate or solvate thereof.

Formula (VII)

In one of its composition aspects, the present embodiments provide a compound of formula (VII):

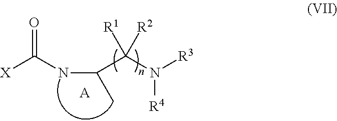

wherein:

X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;

the A ring is a heterocyclic 5 to 12-membered ring;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

n is an integer from 1 to 10;

R³ is selected from alkyl, substituted alkyl, aryl, and substituted aryl;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or a salt, hydrate or solvate thereof.

Formula (VIII)

In one of its composition aspects, the present embodiments provide a compound of formula (VIII):

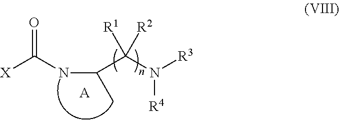

wherein:

X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;

the A ring is a heterocyclic 5 to 12-membered ring;

each R¹ is independently selected from hydrogen, alkyl, substituted alkyl, aryl, substituted aryl, acyl, and aminoacyl;

each R² is independently selected from hydrogen, alkyl, substituted alkyl, aryl, substituted aryl, acyl, and aminoacyl; or

R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;

n is an integer from 1 to 10;

R³ is hydrogen;

R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;

or a salt, hydrate or solvate thereof.

### Certain Embodiments of Formulae I-VIII

In formulae (I)-(IV), X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴.

In certain instances, in formulae (I)-(IV), X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴. In certain instances, in formulae (I)-(IV), X represents a residue of a ketone-containing opioid, wherein the hydrogen atom of the reduced enolic group of the ketone is replaced by a covalent bond to —C(O)—NR⁵—(C(R¹)(R²))ₙ—NR³R⁴. In certain instances, in formulae (I)-(IV), X represents a residue of a ketone-containing opioid, wherein the hydrogen atom of an amino group that is generated from reductive amination of the ketone of the ketone-containing opioid is replaced by a covalent bond to —C(O)—NR⁵—(C(R¹)(R²))ₙ—NR³R⁴.

In formulae (V)-(VIII), X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴.

In certain instances, in formulae (V)-(VIII), X represents a residue of a ketone-containing opioid, wherein the hydrogen atom of the corresponding enolic group of the ketone is replaced by a covalent bond to —C(O)—NR⁵—(C(R¹)(R²))ₙ—NR³R⁴. In certain instances, in formulae (V)-(VIII), X represents a residue of a ketone-containing opioid, wherein the hydrogen atom of the reduced enolic group of the ketone is replaced by a covalent bond to —C(O)—NR⁵—(C(R¹)(R²))ₙ—NR³R⁴. In certain instances, in formulae (V)-(VIII), X represents a residue of a ketone-containing opioid, wherein the hydrogen atom of an amino group that is generated from reductive amination of the ketone of the ketone-containing opioid is replaced by a covalent bond to —C(O)—NR⁵—(C(R¹)(R²))ₙ—NR³R⁴.

In certain instances, X is a ketone-modified opioid drug, wherein the opioid has an optionally substituted morphinan structure and the ketone is situated at the 6-position of the morphinan structure. In certain instances, the opioid has the following optionally substituted morphinan structure:

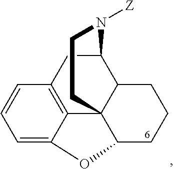

wherein the ketone is situated at the 6-position of the morphinan structure and Z is hydrogen or other group, such as, but not limited to, alkyl or substituted alkyl.

In certain instances, the ketone-containing opioid is attached to the substituent through the enolic oxygen atom of the ketone moiety such that the hydrogen atom of the corresponding enolic group of the ketone-containing opioid is replaced by a covalent bond to the substituent. In certain instances, the ketone-containing opioid is attached to the substituent through the enolic oxygen atom of the ketone moiety such that the hydrogen atom of the corresponding enolic group of the ketone-containing opioid is replaced by a covalent bond to the substituent and the olefin of the corresponding enol is reduced. In certain instances, the ketone-modified opioid is attached to the substituent through an amino group that is generated from reductive amination of the ketone of a ketone-containing opioid.

In certain instances, X is a residue of a ketone-containing opioid selected from acetylmorphone, hydrocodone, hydromorphone, naloxone, N-methylnaloxone, naltrexone, N-methylnaltrexone, oxycodone, oxymorphone, and pentamorphone. In certain instances, X is a residue of a ketone-containing opioid selected from ketobemidone and methadone. In certain instances, X is a residue of a ketone-containing opioid selected from hydrocodone and oxycodone. In certain instances, X is a residue of hydrocodone. In certain instances, X is a residue of oxycodone. In certain instances, X is a residue of a ketone-containing opioid selected from hydromorphone and oxymorphone. In certain instances, X is a residue of hydromorphone. In certain instances, X is a residue of oxymorphone. In certain instances, X is a residue of a ketone-containing opioid selected from naloxone and naltrexone. In certain instances, X is a residue of naloxone. In certain instances, X is a residue of naltrexone. In certain instances, X is a residue of a ketone-containing opioid selected from N-methylnaloxone and N-methylnaltrexone. In certain instances, X is a residue of N-methylnaloxone. In certain instances, X is a residue of N-methylnaltrexone.

In formulae (I)-(IV), R⁵ can be selected from hydrogen, alkyl, substituted alkyl, arylalkyl, substituted arylalkyl, aryl and substituted aryl. In certain instances, R⁵ is hydrogen. In certain instances, R⁵ is (1-6C)alkyl. In other instances, R⁵ is (1-4C)alkyl. In other instances, R⁵ is methyl or ethyl. In other instances, R⁵ is methyl. In certain instances, R⁵ is ethyl.

In certain instances, R⁵ is substituted alkyl. In certain instances, R⁵ is an alkyl group substituted with a carboxylic group such as a carboxylic acid, carboxylic ester or carboxylic amide. In certain instances, R⁵ is —(CH₂)ₙ—COOH, —(CH₂)ₙ—COOCH₃, or —(CH₂)ₙ—COOCH₂CH₃, wherein n is a number form one to 10. In certain instances, R¹ is —(CH₂)₅—COOH, —(CH₂)₅—COOCH₃, or —(CH₂)₅—COOCH₂CH₃.

In certain instances, R⁵ is arylalkyl or substituted arylalkyl. In certain instances, R⁵ is arylalkyl. In certain instances, R⁵ is substituted arylalkyl. In certain instances, R⁵ is an arylalkyl group substituted with a carboxylic group such as a carboxylic acid, carboxylic ester or carboxylic amide. In certain instances, R⁵ is —(CH₂)_{q}(C₆H₄)—COOH, —(CH₂)_{q}(C₆H₄)—COOCH₃, or —(CH₂)_{q}(C₆H₄)—COOCH₂CH₃, where q is an integer from one to 10. In certain instances, R⁵ is —CH₂(C₆H₄)—COOH, —CH₂(C₆H₄)—COOCH₃, or —CH₂(C₆H₄)—COOCH₂CH₃.

In certain instances, R⁵ is aryl. In certain instances, R⁵ is substituted aryl. In certain instances, R⁵ is an aryl group ortho, meta or para-substituted with a carboxylic group such as a carboxylic acid, carboxylic ester or carboxylic amide. In certain instances, R⁵ is —(C₆H₄)—COOH, —(C₆H₄)—COOCH₃, or —(C₆H₄)—COOCH₂CH₃.

In certain instances, in formulae (I)-(IV), when X is a ketone-containing opioid agonist, R⁵ is hydrogen. In certain instances, when X is a ketone-containing opioid antagonist, R⁵ is selected from alkyl, substituted alkyl, arylalkyl, substituted arylalkyl, aryl and substituted aryl.

In formulae (V)-(VIII), the A ring can be a heterocyclic 5 to 12-membered ring.

In certain instances, the A ring is a heterocyclic 5 to 11-membered ring. In certain instances, the A ring is a heterocyclic 5 to 10-membered ring. In certain instances, the A ring is a heterocyclic 5 to 9-membered ring. In certain instances, the A ring is a heterocyclic 5 to 8-membered ring. In certain instances, the A ring is a heterocyclic 5 to 7-membered ring. In certain instances, the A ring is a heterocyclic 5 or 6-membered ring. In certain instances, the A ring is a heterocyclic 5-membered ring.

In certain instances, the A ring is a heterocyclic 6 to 12-membered ring. In certain instances, the A ring is a heterocyclic 6 to 11-membered ring. In certain instances, the A ring is a heterocyclic 6 to 10-membered ring. In certain instances, the A ring is a heterocyclic 6 to 9-membered ring. In certain instances, the A ring is a heterocyclic 6 to 8-membered ring. In certain instances, the A ring is a heterocyclic 6 or 7-membered ring. In certain instances, the A ring is a heterocyclic 6-membered ring. In certain instances, the A ring is a heterocyclic 7-membered ring. In certain instances, the A ring is a heterocyclic 8-membered ring.

In formulae (I)-(VIII), each R¹ can be independently selected from hydrogen, alkyl, substituted alkyl, aryl, substituted aryl, acyl, and aminoacyl. In certain instances, R¹ is hydrogen or alkyl. In certain instances, R¹ is hydrogen. In certain instances, R¹ is alkyl. In certain instances, R¹ is acyl. In certain instances, R¹ is aminoacyl.

In formulae (I)-(VIII), each R² can be independently selected from hydrogen, alkyl, substituted alkyl, aryl, substituted aryl, acyl, and aminoacyl. In certain instances, R² is hydrogen or alkyl. In certain instances, R² is hydrogen. In certain instances, R² is alkyl. In certain instances, R² is acyl. In certain instances, R² is aminoacyl.

In certain instances, R¹ and R² are hydrogen. In certain instances, R¹ and R² on the same carbon are both alkyl. In certain instances, R¹ and R² on the same carbon are methyl. In certain instances, R¹ and R² on the same carbon are ethyl.

In certain instances, R¹ and R¹ which are vicinal are both alkyl and R² and R² which are vicinal are both hydrogen. In certain instances, R¹ and R¹ which are vicinal are both ethyl and R² and R² which are vicinal are both hydrogen. In certain instances, R¹ and R¹ which are vicinal are both methyl and R² and R² which are vicinal are both hydrogen.

In certain instances, in the chain of —[C(R¹)(R²)]ₙ—, not every carbon is substituted. In certain instances, in the chain of —[C(R¹)(R²)]ₙ—, there is a combination of different alkyl substituents, such as methyl or ethyl.

In certain instances, one of R¹ and R² is methyl, ethyl or other alkyl and R⁵ is alkyl. In certain instances, R¹ and R¹ which are vicinal are both alkyl and R² and R² which are vicinal are both hydrogen and R⁵ is alkyl. In certain instances, R¹ and R¹ which are vicinal are both ethyl and R² and R² which are vicinal are both hydrogen and R⁵ is alkyl. In certain instances, R¹ and R¹ which are vicinal are both methyl and R² and R² which are vicinal are both hydrogen and R⁵ is alkyl.

In certain instances, one of R¹ and R² is methyl, ethyl or other alkyl and R⁵ is substituted alkyl. In certain instances, one of R¹ and R² is methyl, ethyl or other alkyl and R⁵ is an alkyl group substituted with a carboxylic group such as a carboxylic acid, carboxylic ester or carboxylic amide. In certain instances, one of R¹ and R² is methyl, ethyl or other alkyl and R⁵ is —(CH₂)_{q}(C₆H₄)—COOH, —(CH₂)_{q}(C₆H₄)—COOCH₃, or —(CH₂)_{q}(C₆H₄)—COOCH₂CH₃, where q is an integer from one to 10. In certain instances, one of R¹ and R² is methyl, ethyl or other alkyl and R⁵ is an alkyl group substituted with carboxamide.

In certain instances, R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group. In certain instances, R¹ and R² together with the carbon to which they are attached can form a cycloalkyl group. Thus, in certain instances, R¹ and R² on the same carbon form a spirocycle. In certain instances, R¹ and R² together with the carbon to which they are attached can form a substituted cycloalkyl group. In certain instances, two R¹ or R² groups on adjacent carbon atoms, together with the carbon atoms to which they are attached, can form a cycloalkyl group. In certain instances, two R¹ or R² groups on adjacent carbon atoms, together with the carbon atoms to which they are attached, can form a substituted cycloalkyl group.

In certain instances, R¹ and R² together with the carbon to which they are attached can form an aryl or substituted aryl group, or two R¹ or R² groups on adjacent carbon atoms, together with the carbon atoms to which they are attached, can form an aryl or substituted aryl group. In certain instances, two R¹ or R² groups on adjacent carbon atoms, together with the carbon atoms to which they are attached, form a phenyl ring. In certain instances, two R¹ or R² groups on adjacent carbon atoms, together with the carbon atoms to which they are attached, form a substituted phenyl ring. In certain instances, two R¹ or R² groups on adjacent carbon atoms, together with the carbon atoms to which they are attached, form a naphthyl ring.

In certain instances, one of R¹ and R² is aminoacyl.

In certain instances, one or both of R¹ and R² is aminoacyl comprising phenylenediamine. In certain instances, one of R¹ and R² is

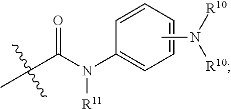

wherein each R¹⁰ is independently selected from hydrogen, alkyl, substituted alkyl, and acyl and R¹¹ is alkyl or substituted alkyl. In certain instances, at least one of R¹⁰ is acyl. In certain instances, at least one of R¹⁰ is alkyl or substituted alkyl. In certain instances, at least one of R¹⁰ is hydrogen. In certain instances, both of R¹⁰ are hydrogen.

In certain instances, one of R¹ and R² is

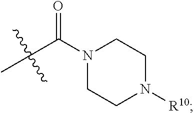

wherein R¹⁰ is hydrogen, alkyl, substituted alkyl, or acyl. In certain instances, R¹⁰ is acyl. In certain instances, R¹⁰ is alkyl or substituted alkyl. In certain instances, R¹⁰ is hydrogen.

In certain instances, one of R¹ and R² is

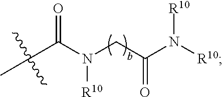

wherein each R¹⁰ is independently hydrogen, alkyl, substituted alkyl, or acyl and b is a number from one to 5. In certain instances, one of R¹ and R² is

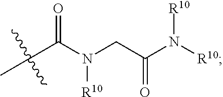

wherein each R¹⁰ is independently hydrogen, alkyl, substituted alkyl, or acyl. In certain instances, one of R¹ and R² is

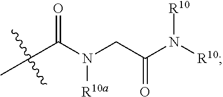

wherein R^{10a} is alkyl and each R¹⁰ is independently hydrogen, alkyl, substituted alkyl, or acyl.

In certain instances, one of R¹ and R² is

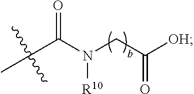

wherein R¹⁰ is independently hydrogen, alkyl, substituted alkyl, or acyl and b is a number from one to 5. In certain instances, one of R¹ and R² is

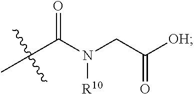

wherein R¹⁰ is independently hydrogen, alkyl, substituted alkyl, or acyl.

In certain instances, one of R¹ and R² is an aminoacyl group, such as —C(O)NR^{10a}R^{10b}, wherein each R^{10a} and R^{10b} is independently selected from hydrogen, alkyl, substituted alkyl, and acyl. In certain instances, one of R¹ and R² is an aminoacyl group, such as —C(O)NR^{10a}R^{10b}, wherein R^{10a} is an alkyl and R^{10b} is substituted alkyl. In certain instances, one of R¹ and R² is an aminoacyl group, such as —C(O)NR^{10a}R^{10b}, wherein R^{10a} is an alkyl and R^{10b} is alkyl substituted with a carboxylic acid or carboxyl ester. In certain instances, one of R¹ and R² is an aminoacyl group, such as —C(O)NR^{10a}R^{10b}, wherein R^{10a} is methyl and R^{10b} is alkyl substituted with a carboxylic acid or carboxyl ester.

In certain instances, R¹ or R² can modulate a rate of intramolecular cyclization. R¹ or R² can speed up a rate of intramolecular cyclization, when compared to the corresponding molecule where R¹ and R² are both hydrogen. In certain instances, R¹ or R² comprise an electron-withdrawing group or an electron-donating group. In certain instances, R¹ or R² comprise an electron-withdrawing group. In certain instances, R¹ or R² comprise an electron-donating group.

Atoms and groups capable of functioning as electron withdrawing substituents are well known in the field of organic chemistry. They include electronegative atoms and groups containing electronegative atoms. Such groups function to lower the basicity or protonation state of a nucleophilic nitrogen in the beta position via inductive withdrawal of electron density. Such groups can also be positioned on other positions along the alkylene chain. Examples include halogen atoms (for example, a fluorine atom), acyl groups (for example an alkanoyl group, an aroyl group, a carboxyl group, an alkoxycarbonyl group, an aryloxycarbonyl group or an aminocarbonyl group (such as a carbamoyl, alkylaminocarbonyl, dialkylaminocarbonyl or arylaminocarbonyl group)), an oxo (═O) substituent, a nitrile group, a nitro group, ether groups (for example an alkoxy group) and phenyl groups bearing a substituent at the ortho position, the para position or both the ortho and the para positions, each substituent being selected independently from a halogen atom, a fluoroalkyl group (such as trifluoromethyl), a nitro group, a cyano group and a carboxyl group. Each of the electron withdrawing substituents can be selected independently from these.

In certain instances, —[C(R¹)(R²)]ₙ— is selected from —CH(CH₂F)CH(CH₂F)—; —CH(CHF₂)CH(CHF₂)—; —CH(CF₃)CH(CF₃)—; —CH₂CH(CF₃)—; —CH₂CH(CHF₂)—; —CH₂CH(CH₂F)—; —CH₂CH(F)CH₂—; —CH₂C(F₂)CH₂—; —CH₂CH(C(O)NR²⁰R²¹)—; —CH₂CH(C(O)OR²²)—; —CH₂CH(C(O)OH)—; —CH(CH₂F)CH₂CH(CH₂F)—; —CH(CHF₂)CH₂CH(CHF₂)—; —CH(CF₃)CH₂CH(CF₃)—; —CH₂CH₂CH(CF₃)—; —CH₂CH₂CH(CHF₂)—; —CH₂CH₂CH(CH₂F)—; —CH₂CH₂CH(C(O)NR²³R²⁴)—; —CH₂CH₂CH(C(O)OR²⁵)—; and —CH₂CH₂CH(C(O)OH)—, in which R²⁰, R²¹, R²² and R²³ each independently represents hydrogen or (1-6C)alkyl, and R²⁴ and R²⁵ each independently represents (1-6C)alkyl.

In formulae (I)-(IV), n can be an integer from 2 to 10. In certain instances, n is two. In other instances, n is three. In other instances, n is four. In other instances, n is five. In other instances, n is six. In other instances, n is seven. In other instances, n is eight. In other instances, n is nine. In other instances, n is ten.

In formulae (V)-(VIII), n is an integer from 1 to 10. In certain instances, n is one. In certain instances, n is two. In other instances, n is three. In other instances, n is four. In other instances, n is five. In other instances, n is six. In other instances, n is seven. In other instances, n is eight. In other instances, n is nine. In other instances, n is ten.

In formula (I) and (V), R³ is selected from hydrogen, alkyl, substituted alkyl, aryl, and substituted aryl. In certain instances, R³ is hydrogen. In certain instances, R³ is alkyl or substituted alkyl. In certain instances, R³ is alkyl. In certain instances, R³ is substituted alkyl. In certain instances, R³ is aryl or substituted aryl. In certain instances, R³ is aryl. In certain instances, R³ is substituted aryl.

In formulae (II), (III), (VI) and (VII), R³ is selected from alkyl, substituted alkyl, aryl, and substituted aryl. In certain instances, R³ is alkyl or substituted alkyl. In certain instances, R³ is alkyl. In certain instances, R³ is substituted alkyl. In certain instances, R³ is aryl or substituted aryl. In certain instances, R³ is aryl. In certain instances, R³ is substituted aryl.

In formulae (IV) and (VIII), R³ is hydrogen.

In formulae (III), (IV), (VII) and (VIII), R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid. The amino acids in the peptide that are not adjacent the nitrogen of —N(R³)(R⁴) can be of D-configuration or L-configuration or a mixture thereof.

In certain instances, in formula (I), R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid. The amino acids in the peptide that are not adjacent the nitrogen of —N(R³)(R⁴) can be of D-configuration or L-configuration.

In certain instances, in formulae (I), (III), (IV), (V), (VII), and (VIII), R⁴ can be a residue of a D-amino acid, a residue of an N-acyl derivative of a D-amino acid, or a residue of a polyethylene glycol derivative of a D-amino acid, wherein the D-amino acid is selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

In certain instances, in formulae (I), (III), (IV), (V), (VII), and (VIII), R⁴ is a residue of D-arginine, D-lysine, D-histidine, D-aspartic acid, or D-glutamic acid or a residue of an N-acyl derivative of D-arginine, D-lysine, D-histidine, D-aspartic acid, or D-glutamic acid, or a residue of a polyethylene glycol derivative of D-arginine, D-lysine, D-histidine, D-aspartic acid, or D-glutamic acid. In certain instances, R⁴ is a residue of D-arginine or D-lysine. In certain instances, R⁴ is a residue of D-arginine. In certain instances, R⁴ is a residue of D-lysine. In certain instances, R⁴ is a residue of D-histidine. In certain instances, R⁴ is a residue of D-aspartic acid. In certain instances, R⁴ is a residue of D-glutamic acid.

In certain instances, in formulae (I), (III), (IV), (V), (VII), and (VIII), R⁴ is a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; or a residue of a polyethylene glycol derivative of a peptide composed of up to five amino acids wherein the amino acid of the peptide adjacent the nitrogen of —N(R³)(R⁴) is a residue of a D-amino acid. The amino acids in the peptide that are not adjacent the nitrogen of —N(R³)(R⁴) can be of D-configuration or L-configuration and can be selected independently from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

In certain instances, in formulae (I), (III), (IV), (V), (VII), and (VIII), in a peptide, N-acyl peptide, or polyethylene glycol of a peptide of R⁴, the amino acid residue adjacent the nitrogen of —N(R³)(R⁴) is a residue of a D-amino acid selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

In certain instances, in formulae (I), (III), (IV), (V), (VII), and (VIII), in a peptide, N-acyl peptide, or polyethylene glycol of a peptide of R⁴, the amino acid residue adjacent the nitrogen of —N(R³)(R⁴) is a residue of D-arginine, D-lysine, D-histidine, D-aspartic acid, or D-glutamic acid. In certain instances, R⁴ is a residue of D-arginine or D-lysine. In certain instances, R⁴ is a residue of D-arginine. In certain instances, R⁴ is a residue of D-lysine. In certain instances, R⁴ is a residue of D-histidine. In certain instances, R⁴ is a residue of D-aspartic acid. In certain instances, R⁴ is a residue of D-glutamic acid.

In certain instances, in formulae (I), (III), (IV), (V), (VII), and (VIII), R⁴ is a monopeptide or an N-acyl derivative or a polyethylene glycol derivative thereof. In certain instances R⁴ is a dipeptide or an N-acyl derivative or a polyethylene glycol derivative thereof. In certain instances R⁴ is a tripeptide or an N-acyl derivative or polyethylene glycol derivative thereof. In certain instances R⁴ is a tetrapeptide or an N-acyl derivative or polyethylene glycol derivative thereof. In certain instances R⁴ is a pentapeptide or an N-acyl derivative or polyethylene glycol derivative thereof.

In certain instances, in formulae (I), (III), (IV), (V), (VII), and (VIII), an acyl derivative is an acetyl, benzoyl, malonyl, piperonyl or succinyl derivative. In certain instances, an acyl derivative is a malonyl derivative. In certain instances, an acyl derivative is a succinyl derivative. In certain instances, an acyl derivative is an acetyl derivative. In certain instances, R⁴ is D-arginyl-N-malonyl, D-lysinyl-N-malonyl, D-arginyl-N-succinyl or D-lysinyl-N-succinyl. In certain instances, R⁴ is D-arginyl-N-acetyl or D-lysinyl-N-acetyl. In certain instances, R⁴ is D-arginyl-N-malonyl. In certain instances, R⁴ is D-lysinyl-N-malonyl. In certain instances, R⁴ is D-arginyl-N-acetyl. In certain instances, R⁴ is D-lysinyl-N-acetyl.

In certain instances, in formulae (I), (III), (IV), (V), (VII), and (VIII), the polyethylene glycol has the following structure:

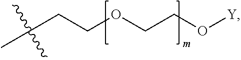

wherein m is a number from one to 20 and Y is hydrogen, alkyl, substituted alkyl, aryl, or substituted aryl.

In formulae (II) and (VI), R⁴ is

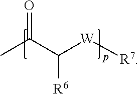

In formula (I) and (V), R⁴ can be

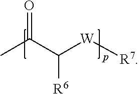

In formulae (I), (II), (V), and (VI), each R⁶ can be independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring.

In certain instances, in formulae (I), (II), (V), and (VI), R⁶ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl. In certain instances, R⁶ is selected from hydrogen, alkyl, substituted alkyl, arylalkyl, substituted arylalkyl, heteroarylalkyl, and substituted heteroarylalkyl. In certain instances, R⁶ is hydrogen. In certain instances, R⁶ is alkyl. In certain instances, R⁶ is substituted alkyl. In certain instances, R⁶ is arylalkyl or substituted arylalkyl. In certain instances, R⁶ is heteroarylalkyl or substituted heteroarylalkyl.

In certain instances, in formulae (I), (II), (V), and (VI), R⁶ is a side chain of an amino acid, such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine or valine. In certain instances, R⁶ is a side chain of arginine, lysine, histidine, aspartic acid, or glutamic acid. In certain instances, R⁶ is a side chain of arginine or lysine. In certain instances, R⁶ is a side chain of arginine. In certain instances, R⁶ is a side chain of lysine. In certain instances, R⁶ is a side chain of histidine. In certain instances, R⁶ is a side chain of aspartic acid. In certain instances, R⁶ is a side chain of glutamic acid.

In certain instances, in formulae (I), (II), (V), and (VI), the R⁶ of R⁴ adjacent the nitrogen of —N(R³)(R⁴) is a side chain of a D-amino acid, and any additional R⁶ can be a side chain of any amino acid independently selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine or valine.

In certain instances, in formulae (I), (II), (V), and (VI), the R⁶ of R⁴ adjacent the nitrogen of —N(R³)(R⁴) is a side chain of a L-amino acid, and any additional R⁶ can be a side chain of any amino acid independently selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine or valine.

In certain instances, in formulae (I), (II), (V), and (VI), the R⁶ of R⁴ adjacent the nitrogen of —N(R³)(R⁴) is a side chain of a D-amino acid or L-amino acid, and any additional R⁶ can be a side chain of any amino acid independently selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine or valine.

In formulae (I), (II), (V), and (VI), each W can be independently —NR⁸—.

In formulae (I), (II), (V), and (VI), each R⁸ can be independently hydrogen, alkyl, substituted alkyl, aryl or substituted aryl, or optionally, each R⁶ and R⁸ independently together with the atoms to which they are bonded form a cycloheteroalkyl or substituted cycloheteroalkyl ring.

In certain instances, in formulae (I), (II), (V), and (VI), R⁸ is hydrogen or alkyl. In certain instances, R⁸ is hydrogen. In certain instances, R⁸ is alkyl. In certain instances, R⁸ is aryl. In certain instances, R⁶ and R⁸ independently together with the atoms to which they are bonded form a cycloheteroalkyl or substituted cycloheteroalkyl ring.

In formulae (I), (II), (V), and (VI), p can be an integer from one to five and each R⁶ can be selected independently from a side chain of any amino acid. In certain instances, p is about 5. In certain instances, p is about 4. In certain instances, p is about 3. In certain instances, p is about 2. In certain instances, p is about one.

In formulae (I), (II), (V), and (VI), R⁷ can be selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol.

In certain instances, R⁷ is hydrogen, alkyl, acyl, or substituted acyl. In certain instances, R⁷ is hydrogen. In certain instances, R⁷ is alkyl. In certain instances, R⁷ is acyl or substituted acyl. In certain instances, R⁷ is acyl. In certain instances, R⁷ is substituted acyl. In certain instances, R⁷ can be acetyl, benzoyl, malonyl, piperonyl or succinyl. In certain instances, R⁷ is malonyl. In certain instances, R⁷ is succinyl. In certain instances, R⁷ is acetyl. In certain instances, R⁷ is polyethylene glycol. In certain instances, R⁷ is polyethylene glycol with the following structure:

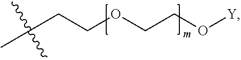

wherein m is a number from one to 20 and Y is hydrogen, alkyl, substituted alkyl, aryl, or substituted aryl.

In formulae (I)-(VIII), the compound comprises a positively charged moiety, a negatively charged moiety or a combination of positively and negatively charged moieties. In certain instances, the compound has at least one moiety that is positively or negatively charged at physiological pH. In certain instances, the compound can have an amino acid with a positive charge, such as arginine, lysine, histidine, or variants thereof. In certain instances, the compound can have an amino acid with a negative charge, such as aspartic acid, glutamic acid, or variants thereof. Additional examples of such moieties include, but are not limited to, guanidines, arylguanidines, amidines, arylamidines, amines, arylamines, carboxylic acids, aryl acids, sulfonic acids, phosphoric acids, or derivatives of any of these moieties.

Particular compounds of interest, and salts or solvates or stereoisomers thereof, include:

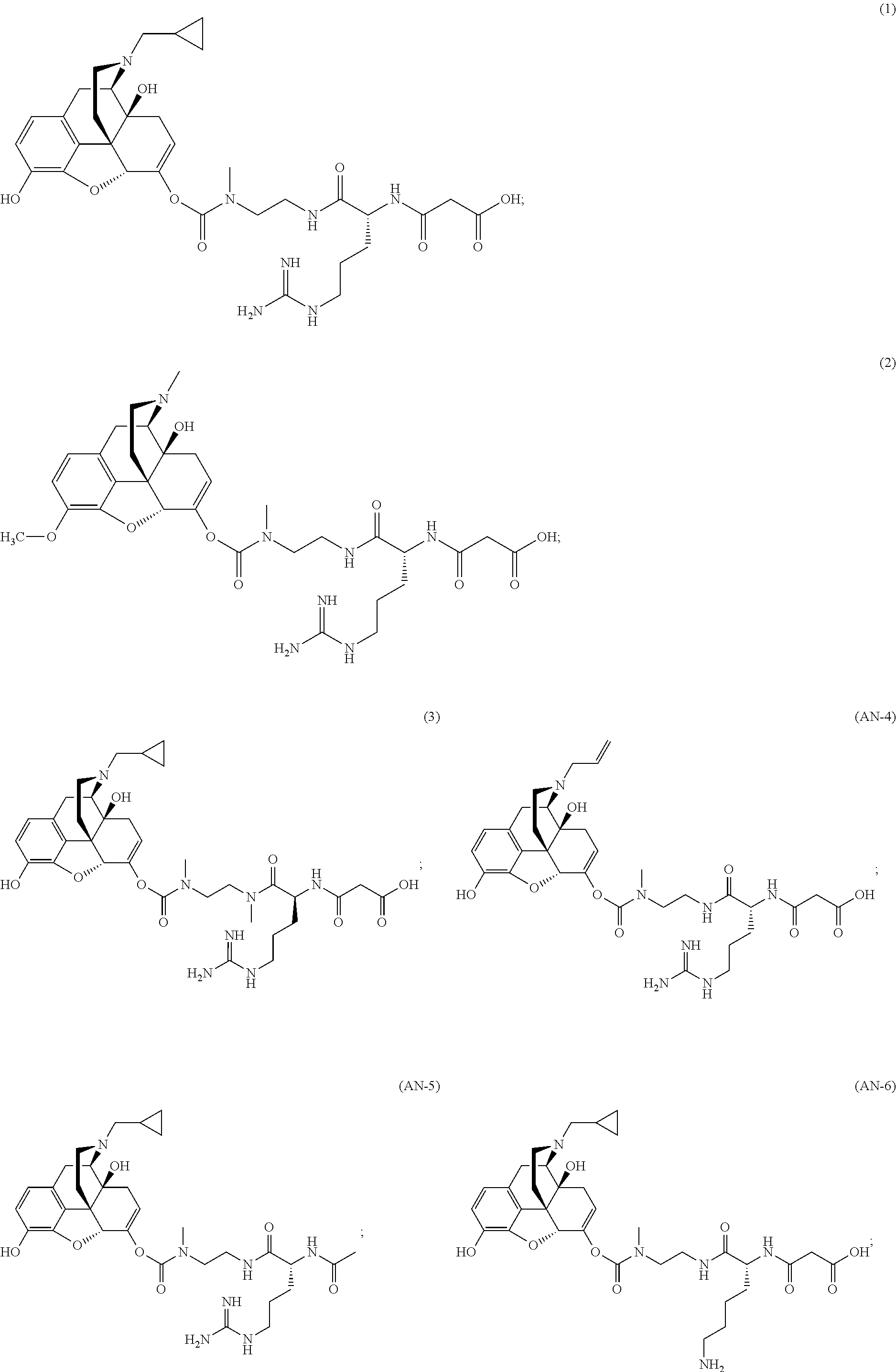

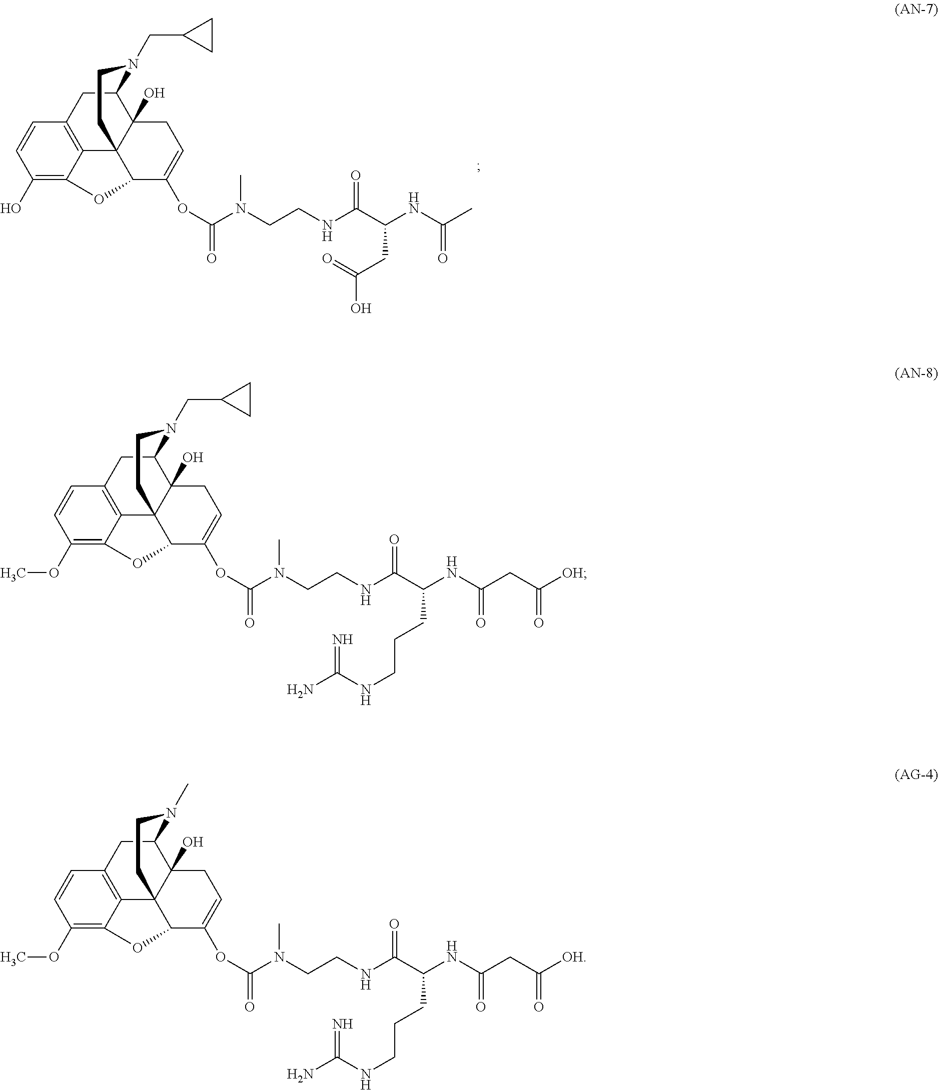

### General Synthetic Procedures for Formulae (I)-(VI)

A representative synthesis for compounds of formulae (I)-(IV) is shown in the following schemes. A representative synthesis for Compound KC203 is shown in Scheme KC-1. In Scheme KC-1, the terms R¹, R², R⁵, and n are defined herein. The terms PG¹ and PG² are amino protecting groups.

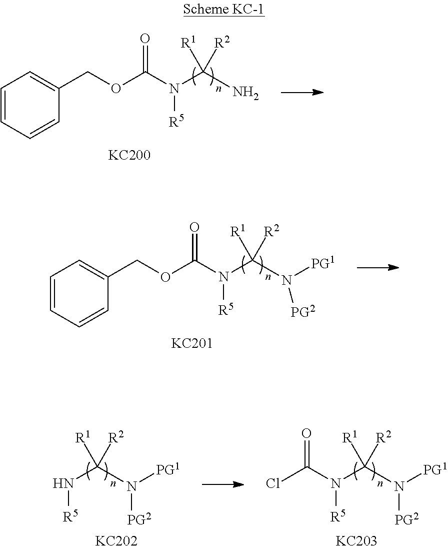

In Scheme KC-1, Compound KC200 is a commercially available starting material. Alternatively, Compound KC200 can be synthesized via a variety of different synthetic routes using commercially available starting materials and/or starting materials prepared by conventional synthetic methods.

With continued reference to Scheme KC-1, Compound KC200 is protected at the amino group to form Compound KC201, wherein PG¹ and PG² are amino protecting groups. Amino protecting groups can be found in T. W. Greene and P. G. M. Wuts, “Protective Groups in Organic Synthesis”, Fourth edition, Wiley, New York 2006. Representative amino-protecting groups include, but are not limited to, formyl groups; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like.

In certain embodiments, PG¹ and PG² are Boc groups. Conditions for forming Boc groups on Compound KC201 can be found in Greene and Wuts, ibid. One method is reaction of Compound KC200 with di-tert-butyl dicarbonate. The reaction can optionally be run in the presence of an activating agent, such as DMAP.

With continued reference to Scheme KC-1, the carboxybenzyl group on Compound KC201 is deprotected to form Compound KC202. Conditions to remove the carboxybenzyl group can be found in Greene and Wuts, ibid. Methods to remove the carboxybenzyl group include hydrogenolysis of Compound KC201 or treatment of Compound KC201 with HBr. One method to remove the carboxybenzyl group is reaction of Compound KC201 with hydrogen and palladium.

With continued reference to Scheme KC-1, Compound KC202 is reacted with phosgene to form Compound KC203. Reaction with phosgene forms an acyl chloride on the amino group of Compound KC202. Other reagents can act as substitutes for phosgene, such as diphosgene or triphosgene.

A representative synthesis for Compound KC302 is shown in Scheme KC-2. In Scheme KC-2, the terms R¹, R², R⁵, and n are defined herein. R^{a} is a substituent on the morphinan ring, such as hydrogen or hydroxyl. The terms PG¹ and PG² are amino protecting groups.

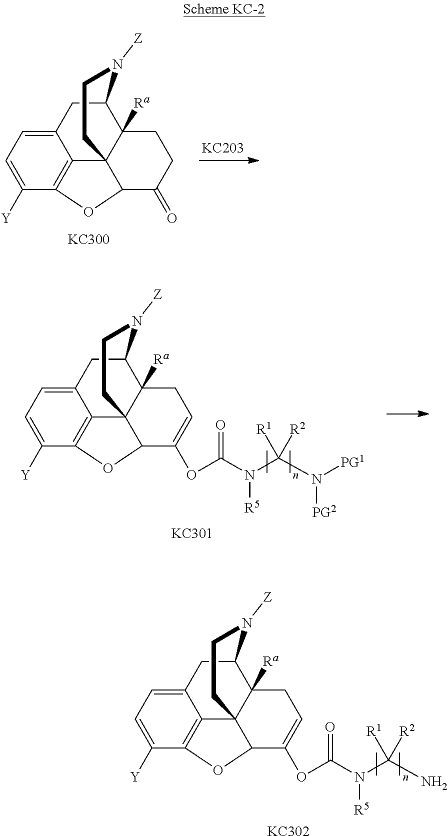

In Scheme KC-2, Compound KC300, where Y is hydroxyl, methoxy, benzyloxy, or silyloxy; Z is methylenecyclopropanyl or methyl; and R^{a} is hydrogen or hydroxyl, is a commercially available starting material. Alternatively, Compound KC300 can be synthesized via a variety of different synthetic routes using commercially available starting materials and/or starting materials prepared by conventional synthetic methods.

With continued reference to Scheme KC-2, Compound KC300 is reacted with Compound KC203 to form Compound KC301. In this reaction, the enolate of Compound KC300 reacts with the acyl chloride of Compound KC203 to form a carbamate.

With continued reference to Scheme KC-2, the protecting groups PG¹ and PG² are removed from Compound KC301 to form Compound KC302. Conditions to remove amino groups can be found in Greene and Wuts. When PG¹ and PG² are Boc groups, the protecting groups can be removed with acidic conditions, such as treatment with trifluoroacetic acid.

A representative synthesis for Compound KC402 is shown in Scheme KC-3. In Scheme KC-3, the terms R^{a}, R¹, R², R⁵, R⁶, R⁷ and n are defined herein. The term PG³ is an amino protecting group.

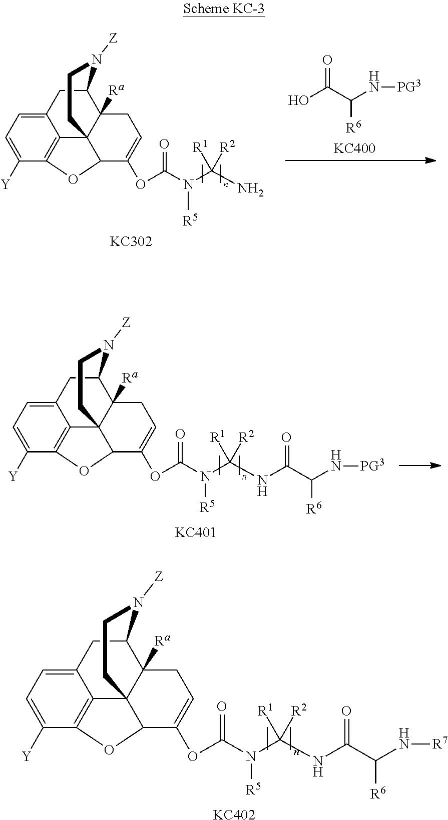

In Scheme KC-3, Compound KC400 is a commercially available starting material. Alternatively, Compound KC400 can be synthesized via a variety of different synthetic routes using commercially available starting materials and/or starting materials prepared by conventional synthetic methods. In certain embodiments, Compound KC400 is reagent in the reaction to give Compound KC402 a residue of a D-amino acid or N-acyl derivative of a D-amino acid. In certain instances, additional amino acid residues can be attached. In such cases, the amino acids in the peptide that are not adjacent the nitrogen of “—N(R³)(R⁴)” can be of D-configuration or L-configuration.

With continued reference to Scheme KC-3, Compound KC302 reacts with Compound KC400 to form Compound KC401 in a peptide coupling reaction. A peptide coupling reaction typically employs a conventional peptide coupling reagent and is conducted under conventional coupling reaction conditions, typically in the presence of a trialkylamine, such as ethyldiisopropylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF. In certain instances, Compound KC302 reacts with Compound KC400 to form Compound KC401 in the presence of HATU and DIEA in DMF.

With continued reference to Scheme KC-3, Compound KC401 is transformed into Compound KC402 with removal of the amino protecting group and addition of an R⁷ group. In certain cases, the amino protecting group is R⁷ and removal of the amino protecting group is optional.

As disclosed herein, representative amino-protecting groups include, but are not limited to, formyl groups; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like. In certain embodiments, PG³ is a Boc group. When PG³ is a Boc group, the protecting group can be removed with acidic conditions, such as treatment with trifluoroacetic acid.

In certain instances, the R⁷ group is added to Compound KC401. Conditions for addition of R⁷ depend on the identity of R⁷ and are known to those skilled in the art. In certain instances R⁷ is an acyl group, such as acetyl, benzoyl, malonyl, piperonyl or succinyl.

N-Acyl derivatives of the compounds of formula KC-(I) may conveniently be prepared by acylating a corresponding compound of formula KC-(I) using an appropriate acylating agent, for example an anhydride, such as acetic anhydride (to prepare an N-acetyl compound) or an acid halide. The reaction is conveniently performed in the presence of a non-reactive base, for example a tertiary amine, such as triethylamine. Convenient solvents include amides, such as dimethyl formamide. The temperature at which the reaction is performed is conveniently in the range of from 0 to 100° C., such as at ambient temperature.

With continued reference to Scheme KC-3, removal of other protecting groups can be performed if other protecting groups were used, such as protecting groups present on the R⁶ moiety. Conditions for removal of other protecting groups depend on the identity of the protecting group and are known to those skilled in the art. The conditions can also be found in Greene and Wuts.

As described in more detail herein, the disclosure provides processes and intermediates useful for preparing compounds of the present disclosure or a salt or solvate or stereoisomer thereof. Accordingly, the present disclosure provides a process of preparing a compound of the present disclosure, the process involves:

contacting a compound of formula:

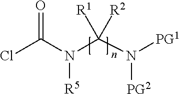

with a compound of formula

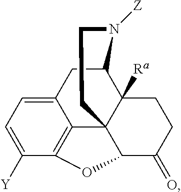

wherein PG¹ and PG² are amino protecting groups; Z is hydrogen, alkyl, or substituted alkyl; and Y is hydroxyl. alkoxy, benzyloxy, or silyloxy.

Accordingly and as described in more detail herein, the present disclosure provides a process of preparing a compound of the present disclosure, the process involves:

contacting a compound of formula:

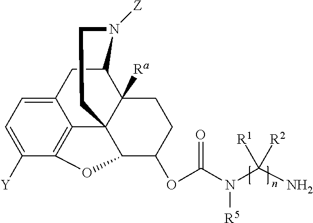

with a compound of formula

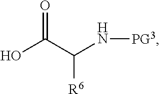

wherein PG³ is an amino protecting group.

In one instance, the above process further involving the step of forming a salt of a compound of the present disclosure. Embodiments are directed to the other processes described herein; and to the product prepared by any of the processes described herein.

### General Synthetic Procedures for Formulae V-VIII

Representative synthetic schemes for compounds disclosed herein are shown below. Compounds of Formulae V-VIII can be synthesized by using the disclosed methods.

A representative synthesis for Compound S-104 is shown in Scheme KC-4. In Scheme KC-4, A ring is defined herein. R^{a} is a substituent on the morphinan ring, such as hydrogen or hydroxyl. PG¹ is an amino protecting group. Although the schemes herein show a morphinan structure for X in Formulae V-VIII, the entire scope of X as a ketone-containing opioid as applicable to Formula is contemplated.

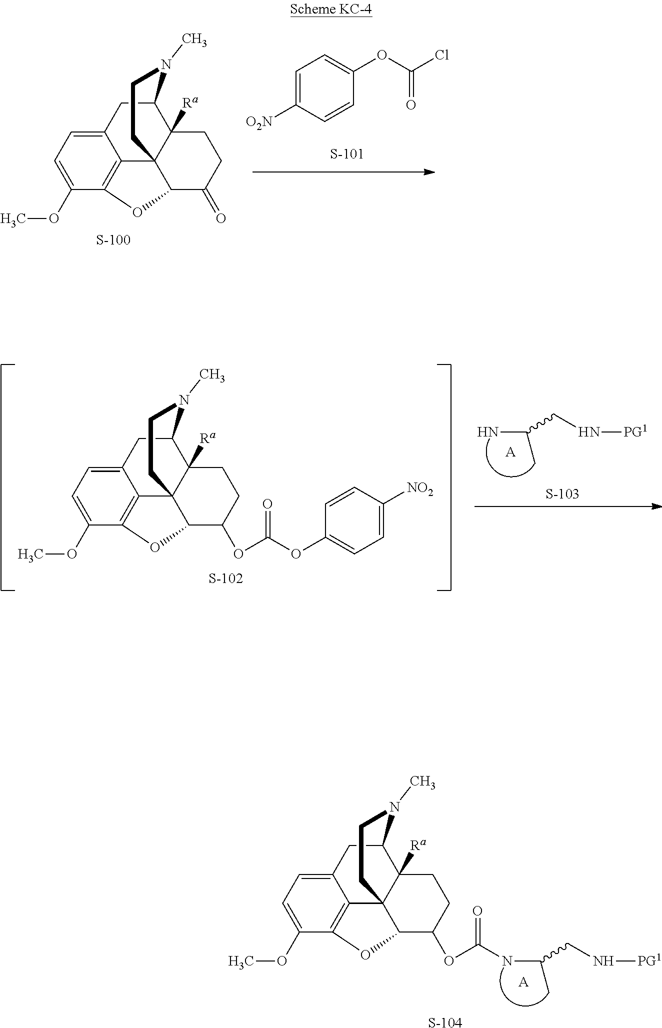

In Scheme KC-4, Compound S-100 is a commercially available starting material. Alternatively, Compound S-100 can be semi-synthetically derived from natural materials or synthesized via a variety of different synthetic routes using commercially available starting materials and/or starting materials prepared by conventional synthetic methods.

With continued reference to Scheme KC-4, Compound S-100 is enolized. Enolization of a ketone can be performed with reaction with a strong base, such as potassium hexamethyldisilazide (KHMDS). The enolate of Compound S-100 is then reacted with an activation agent, such as Compound S-101, to form intermediate Compound S-102. Suitable activation agents include carbonate-forming reagents, such as chloroformates. In Scheme KC-4, the activation agent Compound S-101 is 4-nitrophenyl chloroformate. Other suitable activation agents can be used prior to reaction with Compound S-103.

With continued reference to Scheme KC-4, Compound S-102 reacts with Compound S-103 to form Compound S-104. In Scheme KC-4, Compound S-103 is a commercially available starting material. Alternatively, Compound S-103 can be synthesized via a variety of different synthetic routes using commercially available starting materials and/or starting materials prepared by conventional synthetic methods.

A representative synthesis for Compound S-203 is shown in Scheme KC-5, In Scheme KC-5, R^{a}, A ring and R⁶ are defined herein. PG¹ and PG² are amino protecting groups.

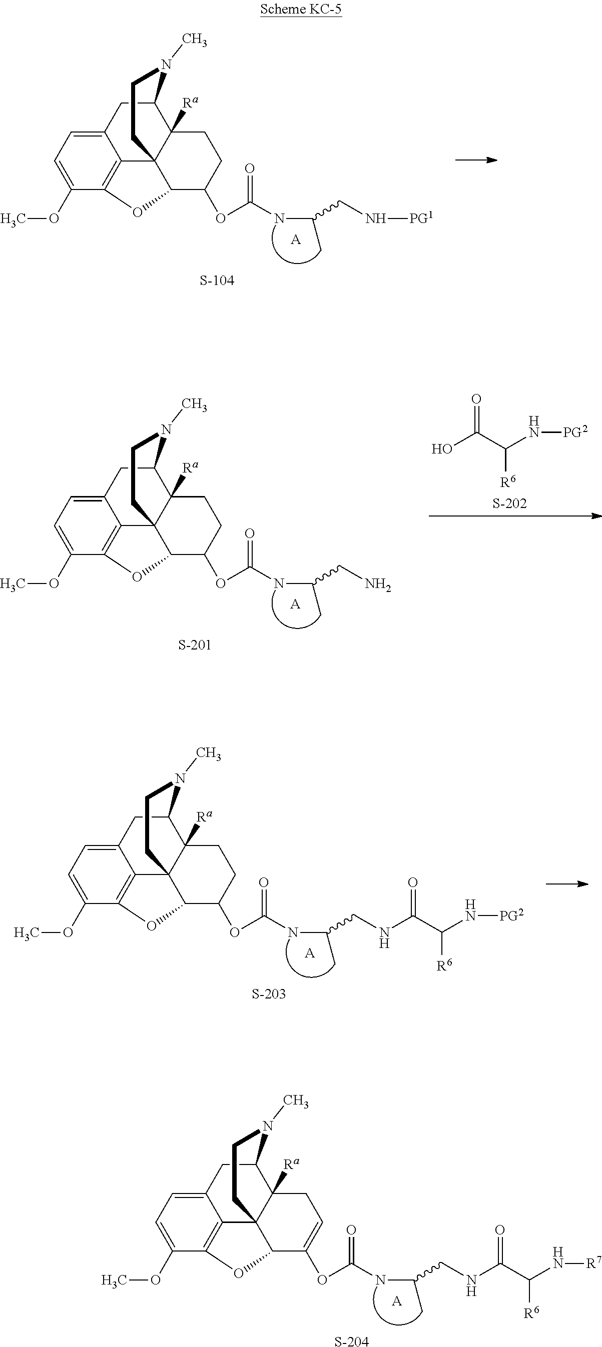

In Scheme KC-5, the protecting group PG¹ is removed from Compound S-104 to form Compound S-201. Conditions to remove amino groups can be found in Greene and Wuts. When PG¹ is a Boc group, the protecting group can be removed with acidic conditions, such as treatment with hydrochloric acid or trifluoroacetic acid.

With reference to Scheme KC-5, Compound S-201 reacts with Compound S-202 to form Compound S-203 in a peptide coupling reaction. In certain embodiments, R⁶ is a side chain of an amino acid and is optionally protected. Protecting groups for the side chain of amino acids are known to those skilled in art and can be found in Greene and Wuts. In certain instances, the protecting group for the side chain of arginine is a sulfonyl-type protecting group, such as 2,2,4,6,7-pentamethyldihydrobenzofurane (Pbf). Other protecting groups include 2,2,5,7,8-pentamethylchroman (Pmc) and 1,2-dimethylindole-3-sulfonyl (MIS).

A peptide coupling reaction typically employs a conventional peptide coupling reagent and is conducted under conventional coupling reaction conditions, typically in the presence of a trialkylamine, such as triethylamine or diisopropylethylamine (DIEA). Suitable coupling reagents for use include, by way of example, carbodiimides, such as ethyl-3-(3-dimethylamino)propylcarbodiimide (EDC), dicyclohexylcarbodiimide (DCC), diisopropylcarbodiimide (DIC) and the like, and other well-known coupling reagents, such as N,N′-carbonyldiimidazole, 2-ethoxy-1-ethoxycarbonyl-1,2-dihydroquinoline (EEDQ), benzotriazol-1-yloxy-tris(dimethylamino)phosphonium hexafluorophosphate (BOP), O-(7-azabenzotriazol-1-yl)-N,N,N,N′,N′-tetramethyluronium hexafluorophosphate (HATU) and the like. Optionally, well-known coupling promoters, such as N-hydroxysuccinimide, 1-hydroxybenzotriazole (HOBT), 1-hydroxy-7-azabenzotriazole (HOAT), N,N-dimethylaminopyridine (DMAP) and the like, can be employed in this reaction. Typically, this coupling reaction is conducted at a temperature ranging from about 0° C. to about 60° C. for about 1 to about 72 hours in an inert diluent, such as THF or DMF. In certain instances, Compound S-201 reacts with Compound S-202 to form Compound S-203 in the presence of HATU.

With continued reference to Scheme KC-5, Compound S-203 is transformed into Compound S-204 with removal of the amino protecting group and addition of an R⁷ group. In certain cases, the amino protecting group is R⁷ and removal of the amino protecting group is optional.

As disclosed herein, representative amino-protecting groups include, but are not limited to, formyl groups; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBS); and the like. In certain embodiments, PG³ is a Boc group. When PG³ is a Boc group, the protecting group can be removed with acidic conditions, such as treatment with trifluoroacetic acid.

In certain instances, the R⁷ group is added to Compound S-203. Conditions for addition of R⁷ depend on the identity of R⁷ and are known to those skilled in the art. In certain instances R⁷ is an acyl group, such as acetyl, benzoyl, malonyl, piperonyl or succinyl.

N-Acyl derivatives of the compounds may conveniently be prepared by acylating a corresponding compound using an appropriate acylating agent, for example an anhydride, such as acetic anhydride (to prepare an N-acetyl compound) or an acid halide. The reaction is conveniently performed in the presence of a non-reactive base, for example a tertiary amine, such as triethylamine. Convenient solvents include amides, such as dimethyl formamide. The temperature at which the reaction is performed is conveniently in the range of from −78° C. to 100° C., such as at ambient temperature.

### Pharmaceutical Compositions and Methods of Use

A composition, such as a pharmaceutical composition, comprises a ketone-modified opioid drug. Such a pharmaceutical composition according to the embodiments can further comprise a pharmaceutically acceptable carrier. The composition is conveniently formulated in a form suitable for oral (including buccal and sublingual) administration, for example as a tablet, capsule, thin film, powder, suspension, solution, syrup, dispersion or emulsion. The composition can contain components conventional in pharmaceutical preparations, e.g. one or more carriers, binders, lubricants, excipients (e.g., to impart controlled release characteristics), pH modifiers, sweeteners, bulking agents, coloring agents or further active agents.

Patients can be humans, and also other mammals, such as livestock, zoo animals and companion animals, such as a cat, dog or horse.

### Pain

In another aspect, the embodiments provide a pharmaceutical composition comprising a ketone-modified opioid drug, such as a ketone-modified opioid agonist drug, as described hereinabove for use in the treatment of pain. A pharmaceutical composition according to the embodiments is useful, for example, in the treatment of a patient suffering from, or at risk of suffering from pain. Accordingly, the present disclosure provides methods of treating or preventing pain in a subject, the methods involving administering to the subject a disclosed composition. The present disclosure provides for a disclosed composition for use in therapy or prevention or as a medicament. The present disclosure also provides the use of a disclosed composition for the manufacture of a medicament, especially for the manufacture of a medicament for the treatment or prevention of pain.

The compositions of the present disclosure can be used in the treatment or prevention of pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

The present disclosure provides use of a ketone-modified opioid drug of formulae (I)-(VIII), such as a ketone-modified opioid agonist drug of formulae (I)-(VIII), in the treatment of pain. The present disclosure provides use of a ketone-modified opioid agonist drug of formulae (I)-(VIII) in the prevention of pain.

The present disclosure provides use of a ketone-modified opioid agonist drug of formulae (I)-(VIII) in the manufacture of a medicament for treatment of pain. The present disclosure provides use of a ketone-modified opioid agonist drug of formulae (I)-(VIII) in the manufacture of a medicament for prevention of pain.

In another aspect, the embodiments provide a method of treating pain in a patient requiring treatment, which comprises administering an effective amount of a pharmaceutical composition as described hereinabove. In another aspect, the embodiments provides method of preventing pain in a patient requiring treatment, which comprises administering an effective amount of a pharmaceutical composition as described hereinabove.

The disclosure provides for a pharmaceutical composition comprising a ketone-modified opioid drug and a further prodrug or drug. Such a prodrug or drug would provide additional analgesia or other benefits. If the composition includes an enzyme-cleavable prodrug, the composition can optionally also include an enzyme inhibitor that interacts with the enzyme(s) that mediates the enzymatically-controlled release of the drug from the prodrug. Examples of suitable further prodrugs or drugs include opioids, acetaminophen, non-steroidal anti-inflammatory drugs (NSAIDs) and other analgesics, as well as prodrugs of any of such drugs. In one embodiment two or more ketone-modified opioid agonist drugs of the embodiments (e.g., a ketone-modified morphine drug and a ketone-modified oxycodone drug), each at a sub-analgesic dose, would be combined to provide a synergistic response leading to effective analgesia with reduced side effects. In one embodiment, two or more opioid agonists, selected from ketone-modified opioid agonists, opioid prodrugs, and/or opioid drugs, each at a sub-analgesic dose, would be combined to provide a synergistic response leading to effective analgesia with reduced side effects. In one embodiment, a ketone-modified opioid antagonist drug would be combined with at least one opioid agonist prodrug or drug. Other examples include drugs or prodrugs that have benefits other than, or in addition to, analgesia. The embodiments provide a pharmaceutical composition, which comprises a ketone-modified opioid drug and acetaminophen. Any combination including acetaminophen can also, but need not necessarily, include an agent that decreases the risk of liver toxicity caused by acetaminophen. Examples of such agents include thiol-containing agents, such as acetylcysteine and methionine. In one embodiment, an example of an agent would be an agent that up-regulates glutathione production in the liver. Also included are pharmaceutically acceptable salts thereof.

### Opioid Antagonist Drugs for Treating and Preventing Unwanted Side Effects

In another aspect, the embodiments provide a pharmaceutical composition, such as a pharmaceutical composition comprising a ketone-modified opioid antagonist drug, as described hereinabove for use in the treatment or prevention of unwanted side effects associated with use of an opioid agonist, particularly an opioid agonist that effects CNS mediated analgesia. Unwanted effects include constipation, cough suppression, dry mouth, heartburn, myocardial depression, nausea, pruritus, urinary retention, vomiting, bloating, dry-mouth and heartburn.

A pharmaceutical composition according to the embodiments is useful, for example, in the treatment of a patient suffering from, or at risk of suffering from unwanted side effects associated with use of an opioid agonist. Accordingly, the present disclosure provides methods of treating or preventing unwanted side effects associated with use of an opioid agonist in a subject, the methods involving administering to the subject a disclosed composition. The present disclosure provides for a disclosed composition for use in therapy or prevention. The present disclosure provides for a disclosed composition for use as a medicament. The present disclosure also provides the use of a disclosed composition for the manufacture of a medicament, especially for the manufacture of a medicament for the treatment or prevention of unwanted side effects associated with use of an opioid agonist.

The present disclosure provides use of a ketone-modified opioid antagonist drug of formulae (I)-(VIII) in the treatment of unwanted side effects associated with use of an opioid agonist. The present disclosure provides use of a ketone-modified opioid antagonist drug of formulae (I)-(VIII) in the prevention of unwanted side effects associated with use of an opioid agonist.

The present disclosure provides use of a ketone-modified opioid antagonist drug of formulae (I)-(VIII) in the manufacture of a medicament for treatment of unwanted side effects associated with use of an opioid agonist. The present disclosure provides use of a ketone-modified opioid antagonist drug of formulae (I)-(VIII) in the manufacture of a medicament for prevention of unwanted side effects associated with use of an opioid agonist.

In another aspect, the embodiments provide a method of treating unwanted side effects associated with use of an opioid agonist in a patient requiring treatment, which comprises administering an effective amount of a pharmaceutical composition as described hereinabove. In another aspect, the embodiments provide a method of preventing unwanted side effects associated with use of an opioid agonist in a patient requiring treatment, which comprises administering an effective amount of a pharmaceutical composition as described hereinabove.

In one embodiment, a ketone-modified opioid antagonist drug can be administered in combination with an opioid agonist or opioid agonist prodrug in order to treat side effects associated with use of such opioid agonist or opioid agonist prodrug in a patient requiring treatment. In one embodiment, a ketone-modified opioid antagonist drug can be administered in combination with an opioid agonist or opioid agonist prodrug in order to prevent side effects associated with use of such opioid agonist or opioid agonist prodrug in a patient requiring treatment.

Such ketone-modified opioid antagonist drug can be co-dosed with such opioid agonist or opioid agonist prodrug or can be administered prior to or following administration of such opioid agonist or opioid agonist prodrug. In certain embodiments, the ketone-modified opioid antagonist drug can be co-dosed with an opioid agonist. In certain embodiments, the ketone-modified opioid antagonist drug can be co-dosed with an opioid agonist prodrug. In certain embodiments, the ketone-modified opioid antagonist drug can be administered prior to or following administration of an opioid agonist. In certain embodiments, the ketone-modified opioid antagonist drug can be administered prior to or following administration of an opioid agonist prodrug.

### Angiogenesis

Angiogenesis is the process by which new blood vessels are formed. Angiogenesis, and the factors that regulate this process, are important in embryonic development, inflammation, and wound healing. Angiogenesis and such regulatory factors also contribute to pathologic conditions such as tumor growth, diabetic retinopathy, rheumatoid arthritis, and chronic inflammatory diseases.

Inappropriate, or pathological, angiogenesis is involved in the growth of atherosclerotic plaque, diabetic retinopathy, degenerative maculopathy, retrolental fibroplasia, idiopathic pulmonary fibrosis, acute adult respiratory distress syndrome, and asthma. Furthermore, tumor progression is associated with neovascularization, which provides a mechanism by which nutrients are delivered to the progressively growing tumor tissue.

In another aspect, the present disclosure provides a pharmaceutical composition comprising a ketone-modified opioid drug, such as a ketone-modified opioid antagonist drug, as described hereinabove, for use in inhibiting angiogenesis, e.g., pathological angiogenesis. The inhibition of angiogenesis, e.g. pathological angiogenesis, can be partial inhibition or complete inhibition. A pharmaceutical composition according to the embodiments is useful, for example, in the treatment of a patient suffering from, or at risk of suffering from, pathological angiogenesis. Accordingly, the present disclosure provides methods of treating or preventing pathological angiogenesis in a subject, the methods involving administering to the subject a disclosed composition. The present disclosure provides for a disclosed composition for use in therapy or prevention or as a medicament. The present disclosure also provides the use of a disclosed composition for the manufacture of a medicament, especially for the manufacture of a medicament for the treatment or prevention of pathological angiogenesis.

A subject method of reducing pathological angiogenesis generally involves administering to an individual in need thereof an effective amount of a subject ketone-modified opioid drug, which can be a ketone-modified opioid antagonist drug.

The present disclosure provides use of a ketone-modified opioid drug of formulae (I)-(VIII) in inhibiting angiogenesis, e.g., pathological angiogenesis. In certain cases, the ketone-modified opioid drug of formulae (I)-(VIII) is a ketone-modified opioid antagonist drug.

The present disclosure provides use of a ketone-modified opioid drug of formulae (I)-(VIII) in the manufacture of a medicament for inhibition of angiogenesis, e.g. pathological angiogenesis. In certain cases, the ketone-modified opioid drug of formulae (I)-(VIII) is a ketone-modified opioid antagonist drug.

In another aspect, the embodiments provide a method of inhibition of angiogenesis, e.g. pathological angiogenesis, in a patient requiring treatment, which comprises administering an effective amount of a ketone-modified opioid drug of formulae (I)-(VIII).

### General Uses of Peripheral Opioid Antagonists

In another aspect, the embodiments provide a pharmaceutical composition, such as a pharmaceutical composition comprising a ketone-modified opioid antagonist drug, as described hereinabove for use in the treatment or prevention of a condition that can be treated or prevented with use of a peripheral opioid antagonist.

A pharmaceutical composition according to the embodiments is useful, for example, in the treatment of a patient suffering from, or at risk of suffering from a condition that can be treated with use of a peripheral opioid antagonist. Accordingly, the present disclosure provides methods of treating or preventing a condition that can be treated or prevented with use of a peripheral opioid antagonist in a subject, the methods involving administering to the subject a disclosed composition. The present disclosure provides for a disclosed composition for use in therapy or prevention. The present disclosure provides for a disclosed composition for use as a medicament. The present disclosure also provides the use of a disclosed composition for the manufacture of a medicament, especially for the manufacture of a medicament for the treatment or prevention of a condition that can be treated or prevented with use of a peripheral opioid antagonist.

The present disclosure provides use of a ketone-modified opioid antagonist drug of formulae (I)-(VIII) in the treatment of a condition that can be treated with use of a peripheral opioid antagonist. The present disclosure provides use of a ketone-modified opioid antagonist drug of formulae (I)-(VIII) in the prevention of a condition that can be prevented with use of a peripheral opioid antagonist.

The present disclosure provides use of a ketone-modified opioid antagonist drug of formulae (I)-(VIII) in the manufacture of a medicament for treatment of a condition that can be treated with use of a peripheral opioid antagonist. The present disclosure provides use of a ketone-modified opioid antagonist drug of formulae (I)-(VIII) in the manufacture of a medicament for prevention of a condition that can be prevented with use of a peripheral opioid antagonist.

In another aspect, the embodiments provide a method of treating a condition that can be treated with use of a peripheral opioid antagonist in a patient requiring treatment, which comprises administering an effective amount of a pharmaceutical composition as described hereinabove. In another aspect, the embodiments provides method of preventing a condition that can be prevented with use of a peripheral opioid antagonist in a patient requiring treatment, which comprises administering an effective amount of a pharmaceutical composition as described hereinabove.

### Administration of Pharmaceutical Compositions

The amount of composition disclosed herein to be administered to a patient to be effective (i.e. to provide blood levels of ketone-containing opioid sufficient to be effective in the treatment or prophylaxis of pain or of the side effects of an opioid agonist) will depend upon the bioavailability of the particular composition as well as other factors, such as the species, age, weight, sex, and condition of the patient, manner of administration and judgment of the prescribing physician. In general, the dose can be such that the ketone-modified opioid drug is in the range of from 0.01 milligrams per kilogram to 20 milligrams drug per kilogram (mg/kg) body weight. For example, a ketone-modified opioid drug can be administered at a dose in the range of from 0.02 to 0.5 mg/kg body weight or 0.01 mg/kg to 10 mg/kg body weight or 0.01 to 2 mg/kg body weight. In one embodiment, a ketone-modified opioid drug can be administered at a dose in the range of 0.01 mg/kg to 15 mg/kg body weight. In one embodiment, the composition can be administered at a dose such that the level of ketone-modified opioid drug achieved in the blood is in the range of from 0.5 ng/ml to 200 ng/ml.

The disclosure provides for a pharmaceutical composition comprising a ketone-modified opioid drug and a further prodrug or drug. Such a prodrug or drug would provide additional analgesia or other benefits. If the composition includes an enzyme-cleavable prodrug, the composition can optionally also include an enzyme inhibitor that interacts with the enzyme(s) that mediates the enzymatically-controlled release of the drug from the prodrug. Examples include opioids, acetaminophen, non-steroidal anti-inflammatory drugs (NSAIDs) and other analgesics. In one embodiment, a ketone-modified opioid antagonist drug would be combined with an opioid agonist prodrug or drug. Other examples include drugs or prodrugs that have benefits other than, or in addition to, analgesia. The embodiments provide a pharmaceutical composition, which comprises ketone-modified opioid drug and acetaminophen. Also included are pharmaceutically acceptable salts thereof.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Example 1

### Syntheses of N-(naltrexone-6-enol-carbonyl-methyl amino)ethylamine-D-arginine-malonic acid (Compound AN-1; also referred to Compound 1, and as N-{(S)-4-guanidino-1-[2-(methyl-[17-(cyclopropylmethyl)-4,5α-epoxy-3,14-dihydroxymorphinan-6-oxy]carbonyl-amino)-ethylcarbamoyl]-butyl}-malonic acid) and N-(oxycodone-6-enol-carbonyl-methyl amino)ethylamine-D-arginine-malonic acid (Compound AG-2; also referred to Compound 2, and as N-{(S)-4-guanidino-1-[2-(methyl-[(5R,9R,13S,14S)-4,5a-epoxy-6,7-didehydro-14-hydroxy-3-methoxy-17-methylmorphinan-6-oxy]carbonyl-amino)-ethylcarbamoyl]-butyl}-malonic acid)

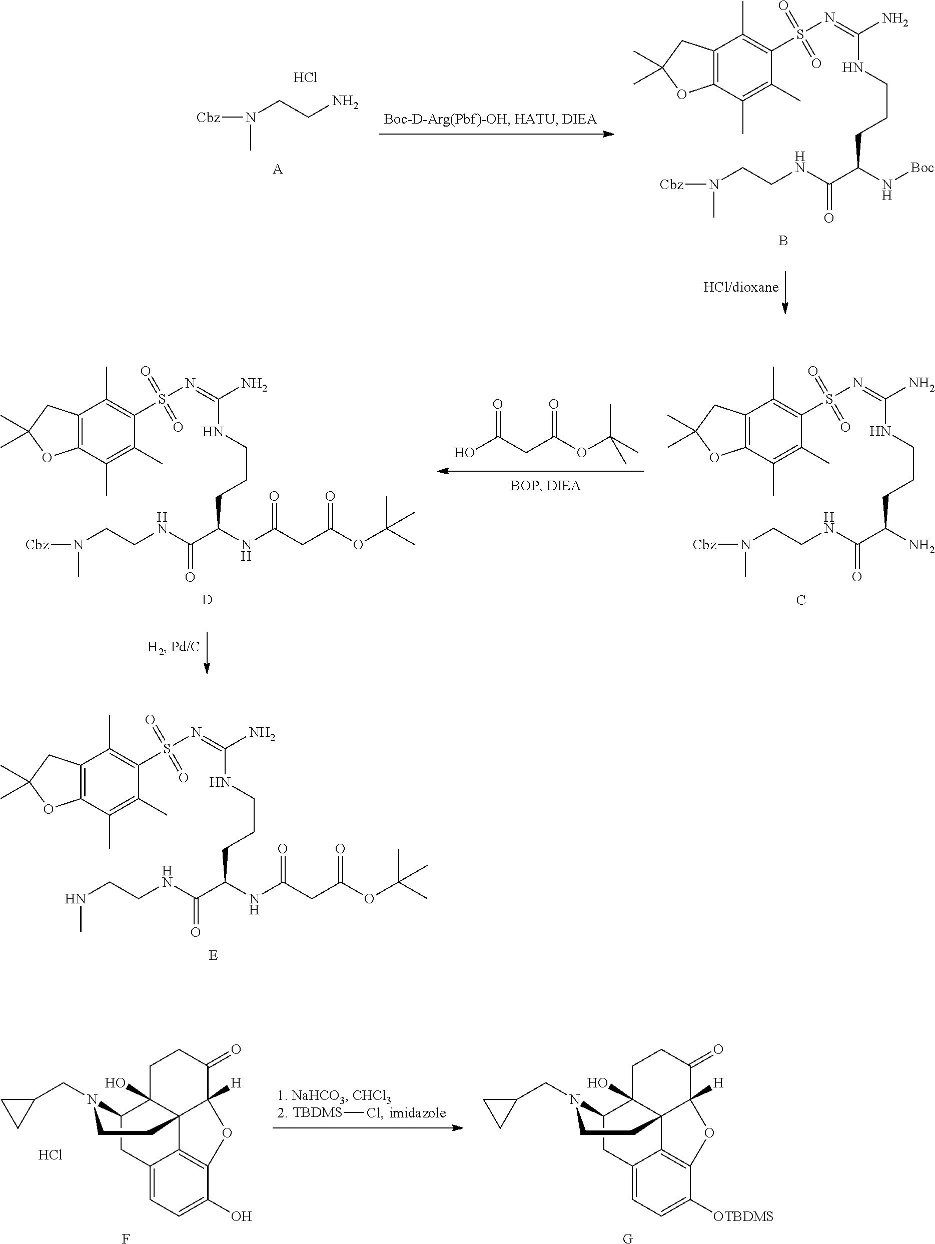

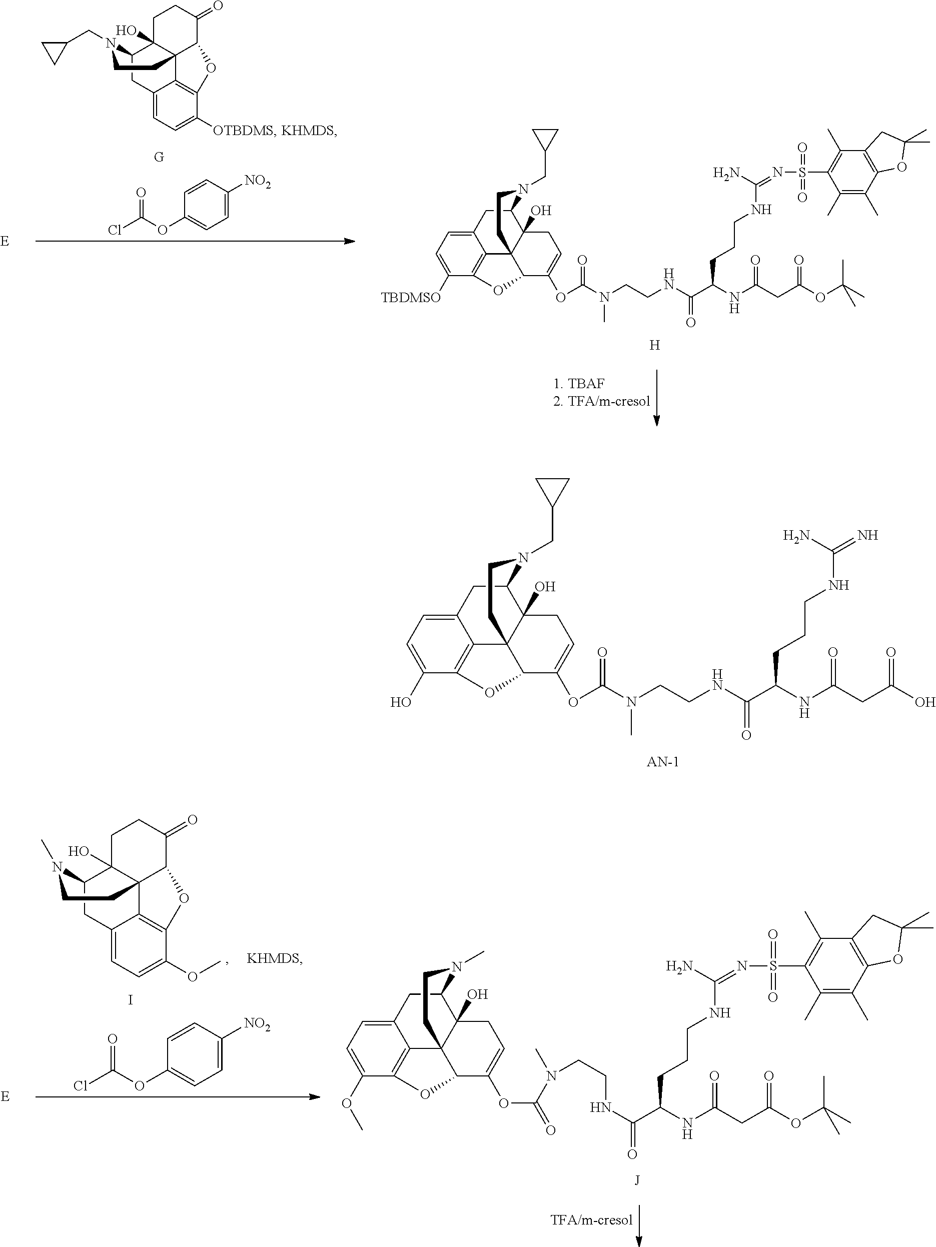

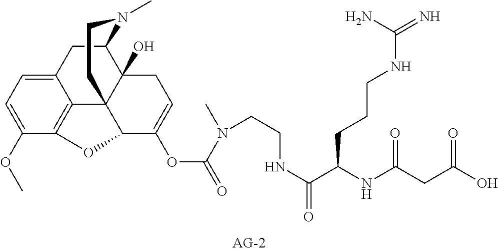

### Preparation of Compound B

To a cooled solution (˜5° C.) of Boc-(D)-Arg(Pbf)-OH (24.3 g, 46.1 mmol), Compound A hydrochloride (11.9 g, 48.5 mmol) and HATU (19.9 g, 52.4 mmol) in DMF (250 mL) was added DIEA dropwise (38.3 mL, 218 mmol) over 30 min. After complete addition, the ice bath was removed and the mixture stirred at ambient temperature for 30 min. Most of the DMF was removed in vacuo and the residue taken into EtOAc (800 mL) and washed with water (600 mL), 2% aq. H₂SO₄ (100 mL), water (3×600 mL) and brine (600 mL). The organic layer was dried over MgSO₄, filtered, and the solvent evaporated to provide compound B (33.8 g, yield exceeded quantitative) as an off-white foamy solid. LC-MS [M+H] 717.4 (C₃₅H₅₂N₆O₈S+H, calc: 717.9). Compound B was used in the next step without further purification.

### Preparation of Compound C

A solution of compound B (33.8 g, ˜46.1 mmol) in DCM (140 mL) was treated with 4 M HCl in dioxane (138 mL, 552 mmol) and stirred at ambient temperature for 30 min. All volatiles were removed and the residue dried in vacuo to give compound C as a light-brownish foamy solid (35.5 g, 51.5 mmol, yield exceeded quantitative). LC-MS [M+H] 617.7 (C₃₀H₄₄N₆O₆S+H, calc: 617.9). Compound C was used in the next step without further purification.

### Preparation of Compound D

To a cooled solution (˜5° C.) of compound C (35.5 g, ˜46.1 mmol) and mono tert-butyl malonate (7.75 g, 48.4 mmol, 7.16 mL) in DMF (170 mL) was added BOP (21.8 g, 49.3 mmol), followed by DIEA (24.0 g, 184 mmol, 32.3 mL) dropwise over 25 min. After complete addition, the ice bath was removed and the mixture stirred at ambient temperature. After 30 min, most of the DMF was evaporated in vacuo and the residue taken into EtOAc (1 L) and washed with water (800 mL), 2% aq. H₂SO₄ (100 mL), water (5×500 mL) and brine (2×500 mL). After drying over MgSO₄, the solvent was evaporated in vacuo to give crude compound D (38 g, yellow oil), which was purified by column chromatography (SiO₂ 330 g, 0-10% MeOH gradient in DCM). Pure compound D (23.2 g, 30.5 mmol, 66% over 3 steps) was isolated as an off-white foamy solid. LC-MS [M+H] 759.4 (C₃₇H₅₄N₆O₉S+H, calc: 759.9). TLC R_{f} (DCM/MeOH 95:5): 0.14.

### Preparation of Compound E

Compound D (23.2 g, 30.5 mmol) was dissolved in methanol (180 mL) followed by the addition of a Pd/C (5% wt, 4.0 g) suspension in water (10 mL). The reaction mixture was subjected to hydrogenation (Parr apparatus, 70 psi H₂) at ambient temperature for 1 h. The mixture was filtered over celite and washed with methanol (100 mL). The filtrate was concentrated in vacuo and rinsed with toluene (2×50 mL). Drying in vacuo gave compound E (19.0 g, 30.5 mmol, 100%) as an off-white foamy solid. LC-MS [M+H] 625.5 (C₂₉H₄₈N₆O₇S+H, calc: 625.8). Compound E was used in the next step without further purification.

### Preparation of Naltrexone Free Base

Naltrexone hydrochloride, Compound F (13.8 g, 37.9 mmol) was dissolved in water (200 mL), brought to pH 8 by addition of saturated NaHCO₃ solution and extracted with chloroform (6×125 mL). The combined organic layers were washed with brine (350 mL), dried over MgSO₄ and concentrated. Traces of water were removed by dissolving the residue in toluene (2×50 mL) and evaporating it. After drying, naltrexone free base (11.8 g, 36.0 mmol, 95%) was recovered as a white solid.

### Preparation of Compound G

To a cooled (˜5° C.) solution of Naltrexone free base (11.8 g, 36.0 mmol) in DMF (110 mL) were added imidazole (3.68 g, 54 mmol) and TBDMS-Cl (5.43 g, 36.0 mmol). Ten min after complete addition the bath was removed and the mixture stirred at ambient temperature for 16 h. Most of the DMF was removed in vacuo and the residue taken into EtOAc (600 mL), washed with water (2×500 mL) and brine (300 mL) and dried over MgSO₄. After evaporation of the solvent in vacuo, the crude material (white solid, 15 g) was purified by column chromatography (SiO₂ 330 g, 100% hexane to remove excess of TBDMS-Cl (if any), followed by gradient 0-80% EtOAc in hexane). Pure compound G (12.9 g, 28.4 mmol, 79%) was isolated as a white solid. LC-MS [M+H] 456.5 (C₂₆H₃₇NO₄Si+H, calc: 456.7). TLC R_{f} (EtOAc/hexane 4:6): 0.3.

### Preparation of Compound H

To a cooled (−78° C.) solution of G (5.31 g, 11.7 mmol) in anhydrous THF (200 mL) was added, under N₂, dropwise a 0.5 M solution of KHMDS in toluene over 25 min. The yellow solution was stirred at this temperature for 30 min. Then, the solution was added through a metal cannula to a cooled solution (−78° C.) of 4-nitrophenyl chloroformate (2.35 g, 11.7 mmol) in anhydrous THF (50 mL) over 5 min. Stirring at −78° C. was continued until the solution was treated dropwise with a solution of compound E (4.04 g, 6.47 mmol) in anhydrous THF (25 mL) over 30 min. After 1.5 h at −78° C., the reaction mixture was quenched with saturated NaHCO₃ solution (10 mL). The precipitate was filtered and washed with EtOAc (30 mL). The residue was taken into EtOAc (500 mL), washed with water (3×350 mL) and brine (300 mL) and dried (MgSO₄). After evaporation of the solvent, the residue was dried in vacuo to give compound H (10.5 g, yield exceeded quantitative) as a yellow foamy solid. LC-MS [M+H] 1106.5 (C₅₆H₈₃N₇O₁₂SSi+H, calc: 1107.5). Compound H was used in the next step without further purification.

### Synthesis of N-(naltrexone-6-enol-carbonyl-methyl amino)ethylamine-D-arginine-malonic acid (Compound AN-1)

To a cooled (˜5° C.) solution of compound H (10.5 g, ˜6.47 mmol) in anhydrous THF (30 mL) was added dropwise a 1 M solution of TBAF in THF (9.7 mmol, 9.7 mL) over 20 min. After complete addition the bath was removed and the reaction mixture stirred at ambient temperature for 30 min, diluted with EtOAc (400 mL) and washed with water (3×400 mL) and brine (300 mL). Drying over MgSO₄, evaporation of the solvent and drying the residue in vacuo gave TBDMS-deprotected compound H (8.47 g, >100%).

A solution of TBDMS-deprotected compound H (8.47 g, ˜6.47 mmol) in 5% m-cresol/TFA (200 mL) was stirred at ambient temperature. After 30 min, the mixture was diluted with ether (1 L). The resulting fine suspension was filtered, the solid washed with ether (50 mL) and hexane (50 mL) and dried in vacuo for 15 min. This crude material (10 g) was split into 3 portions and each dissolved in a 1:6-mixture of AcOH:water (40 mL) and purified by HPLC [Nanosyn-Pack Microsorb (100-10) C-18 column (50×300 mm); flow rate: 100 mL/min; injection volume: 40 mL; mobile phase A: 100% water, 0.1% TFA; mobile phase B: 100% acetonitrile, 0.1% TFA; gradient elution 0% B in 2 min, gradient elution from 0% B to 10% B in 15 min, isocratic elution at 10% B in 30 min, gradient elution from 10% B to 33% B in 48 min; detection at UV 254 nm]. Fractions containing the desired compound were combined and concentrated in vacuo. The residue was treated with toluene (2×50 mL) and co-evaporated in vacuo (procedure repeated twice). The residue was then dissolved in acetonitrile (10 mL), treated with 2.0 M HCl in ether (200 mL), followed by dilution with ether (600 mL). The resulting solid was filtered, washed with ether (50 mL) and hexane (50 mL) and dried in vacuo overnight to provide Compound AN-1, also referred to as Compound 1 (2.03 g, 40% yield over 3 steps) as a white solid, hydrochloride salt. LC-MS [M+H] 684.4 (C₃₃H₄₅N₇O₉+H, calc: 684.8). Purity >95% (UV/254 nm). Retention time [Chromolith SpeedRod RP-18e C18 column (4.6×50 mm); flow rate 1.5 mL/min; mobile phase A: 0.1% TFA/water; mobile phase B 0.1% TFA/ACN; gradient elution from 5% B to 100% B over 9.6 min, detection 254 nm]: 2.16 min.

### Preparation of Oxycodone Free Base (Compound I)

Oxycodone hydrochloride (10.0 g, 28.5 mmol) was dissolved in chloroform (150 mL) and washed with 5% aq. NaHCO₃ (50 mL). The organic layer was removed, dried over MgSO₄ and evaporated. The residue was dried in vacuo overnight to provide oxycodone free base, compound I, (8.3 g, 93%) as a white solid.

### Preparation of Compound J

To a cooled (−78° C.) solution of oxycodone free base, compound I, (3.65 g, 11.6 mmol) in anhydrous THF (200 mL) was added, under N₂, dropwise a 0.5 M solution of KHMDS in toluene over 30 min. The yellow solution was stirred at this temperature for 30 min. The solution was then added through a metal cannula to a cooled solution (−78° C.) of 4-nitrophenyl chloroformate (2.33 g, 11.6 mmol) in anhydrous THF (50 mL) over 5 min. Stirring at −78° C. was continued until the solution was treated dropwise with a solution of compound E (4.01 g, 6.42 mmol) in anhydrous THF (30 mL) over 30 min. After complete addition, the bath was removed and the reaction mixture was stirred at ambient temperature for 1 h. The reaction mixture was quenched with saturated NaHCO₃ solution (10 mL). The precipitate was filtered and washed with EtOAc (30 mL). The residue was taken into EtOAc (500 mL), washed with water (3×350 mL) and brine (300 mL) and dried (MgSO₄). After evaporation of the solvent, the residue was dried in vacuo to give compound J (8.57 g, yield exceeded quantitative) as a yellow foamy solid. LC-MS [M+H] 966.9 (C₄₈H₆₇N₇O₁₂S+H, calc: 967.2). Compound J was used in the next step without further purification.

### Synthesis of N-(oxycodone-6-enol-carbonyl-methyl amino)ethylamine-D-arginine-malonic acid (Compound AG-2)

A solution of compound J (8.57 g, ˜6.4 mmol) in 5% m-cresol/TFA (200 mL) was stirred at ambient temperature. After 30 min, the mixture was diluted with ether (1 L). The resulting fine suspension was filtered, the solid washed with ether (50 mL) and hexane (50 mL) and dried in vacuo for 15 min. This crude material (6 g) was split into 3 portions and each dissolved in a 1:6-mixture of AcOH:water (40 mL) and purified by HPLC [Nanosyn-Pack Microsorb (100-10) C-18 column (50×300 mm); flow rate: 100 mL/min; injection volume: 40 mL; mobile phase A: 100% water, 0.1% TFA; mobile phase B: 100% acetonitrile, 0.1% TFA; gradient elution 0% B in 2 min, gradient elution from 0% B to 10% B in 15 min, isocratic elution at 10% B in 30 min, gradient elution from 10% B to 33% B in 48 min; detection at UV 254 nm]. Fractions containing the desired compound were combined and concentrated in vacuo. The residue was treated with toluene (2×50 mL) to remove traces of water and co-evaporated in vacuo (procedure repeated twice). The residue was dissolved in acetonitrile (10 mL), treated with 2.0 M HCl in ether (200 mL), followed by dilution with ether (600 mL). The resulting solid was filtered, washed with ether (50 mL) and hexane (50 mL) and dried in vacuo overnight to provide Compound AG-2, also referred to as Compound 2 (2.44 g, 3.18 mmol, 50% yield over 2 steps) as a white powdery solid, hydrochloride salt. LC-MS [M+H] 658.6 (C₃₁H₄₃N₇O₉+H, calc: 658.7). Purity >95% (UV/254 nm). Retention time [Chromolith SpeedRod RP-18e C18 column (4.6×50 mm); flow rate 1.5 mL/min; mobile phase A: 0.1% TFA/water; mobile phase B 0.1% TFA/ACN; gradient elution from 5% B to 100% B over 9.6 min, detection 254 nm]: 2.22 min.

### Example 2

### Synthesis of N-methyl-N-(naloxone-6-enol-carbonyl-methyl amino)ethylamine-arginine-malonic acid (Compound AN-3; also referred to as Compound 3, and as N-[1-({2-[(5R,9R,13S,14S)-4,5a-epoxy-6,7-didehydro-3,14-dihydroxy-3-17-cyclopropylmethylmorphinan-6-oxy]-1-enyloxycarbonyl)-methyl-amino]-ethyl}-methyl-carbamoyl)-4-guanidino-butyl]-malonic acid) **[Connie: All of the following opioid compounds needed the appropriate stereochemistry. OC's bridge, the tertiary alcohol and the ether near the ketone. (see previous compounds for examples. Also, removed the “D” in Boc-D-Arg(Pbf)-OH, this compound in particular is not “D”]

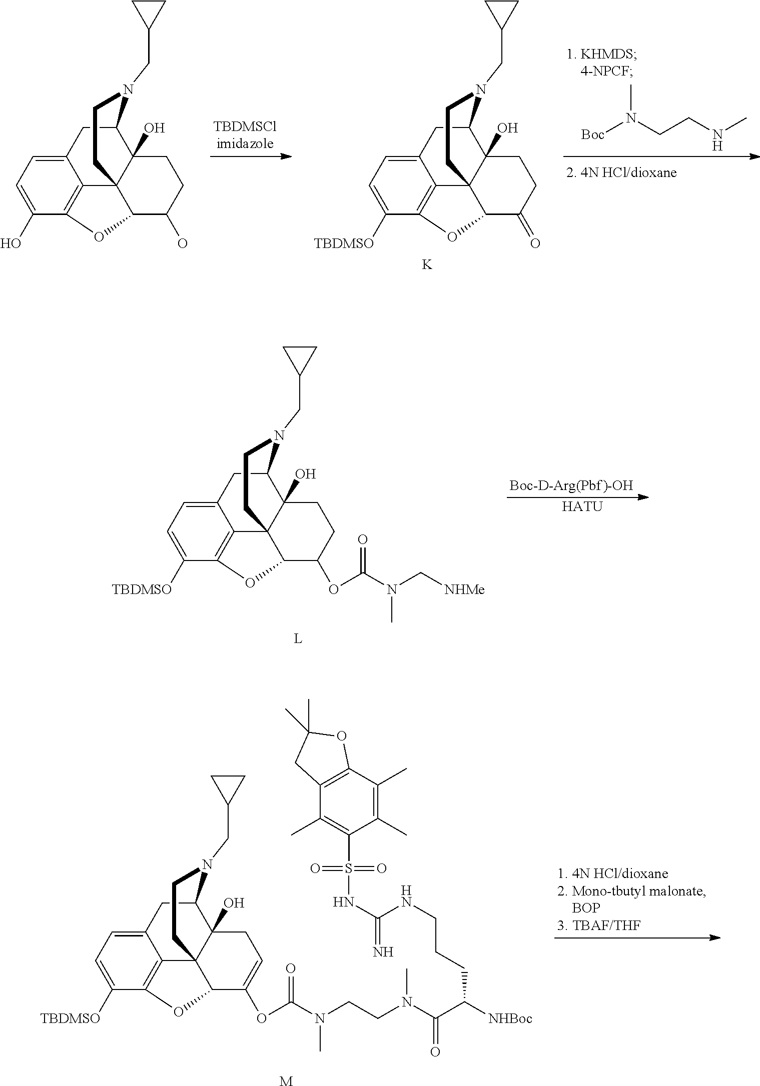

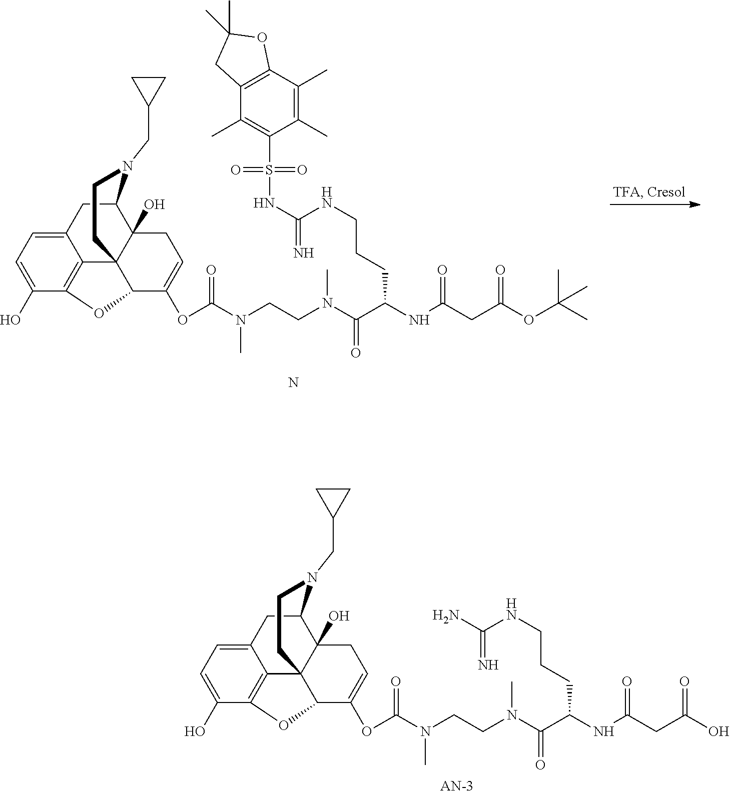

### Preparation of Compound K

Naltrexone free base (5.0 g, 13.2 mmol) was dissolved in dry DMF (40 mL) at ambient temperature. Imidazole (1.35 g, 19.9 mmol) was added in one portion, followed by TBDMSCl (2.0 g, 13.2 mmol). The mixture was stirred at ambient temperature for 15 h. The volatiles were then removed in vacuo. The residue was partitioned between DCM (250 mL) and water (250 mL). The organic layer was washed with brine (250 mL) and dried over Na₂SO₄. The mixture was then filtered and concentrated to afford compound K as a white solid (5.43 g, 11.9 mmol, 90%). LC-MS [M+H] 457.0 (C₂₆H₃₇NO₄Si+H, calc: 457.0). Compound K was used in the next step without further purification.

### Preparation of Compound L

Compound K (5.9 g, 13.2 mmol) was dissolved in dry THF (degassed) (78 mL) and the mixture was cooled to −78° C. using a dry ice/acetone bath. KHMDS (26 mL, 52.0 mmol, 0.5 M in toluene) was added via syringe. The mixture was stirred at −78° C. for 30 min. In a separate flask were dissolved 4-nitro-phenylchloroformate (4-NPCF) (2.6 g, 12.9 mmol) and THF (20 mL). This mixture was chilled to −78° C. using a dry ice/acetone bath. The mixture from intermediate compound K was then transferred via cannula to the second flask containing the 4-NPCF solution over ˜5 min. The mixture was further stirred at −78° C. for 30 min and then warmed up to −10° C. Methyl-(2-methylamino-ethyl)-carbamic acid tert-butyl ester (2.43 g, 12.9 mmol) in THF (10 mL) was added. The mixture was stirred at ambient temperature for 15 h. NaHCO₃ (2 mL, sat. aq.) was then added. The mixture was concentrated in vacuo and EtOAc (50 mL) was added, followed by water (40 mL). The layers were separated, and the organic layer was further washed with water (20 mL) and brine (20 mL). The organic layer was then concentrated, and the residue was purified by silica gel chromatography, using DCM/MeOH (gradient 100/1 to 100/15) to afford the product as a colorless oil (7 g, 10.4 mmol). This material was dissolved in DCM (10 mL) at ambient temperature and treated with 4N HCl in dioxane (20 mL). The mixture was stirred for 2 h. The mixture was then concentrated in vacuo to afford crude amine compound L as a white HCl salt (˜6.7 g, 10.6 mmol, 80%). LC-MS [M+H] 571.2 (C₃₁H₄₇N₃O₅Si+H, calc: 571.3). This mixture contained some amount of by-product from loss of the phenolic TBDMS group. The mixture was used in the next step without purification.

### Preparation of Compound M

Compound L (6.7 g, ˜10.4 mmol) was dissolved in DMF (100 mL). Boc-(L)-Arg(Pbf)-OH (5.5 g, 10.44 mmol), HATU (4.4 g, 11.48 mmol) and DIEA (5.5 mL, 31.6 mmol) were added in this order. The reaction was continued at ambient temperature for 2 h. The mixture was then concentrated, and the residue was partitioned between EtOAc and water (30 mL/20 mL). The organic layer was removed and then washed with water (20 mL), brine (20 mL), dried over Na₂SO₄ and concentrated thoroughly to afford crude compound M. LC-MS [M+H] 1079.9 (C₅₅H₈₃N₇O₁₁SSi+H, calc: 1079.5). Compound M was used in the next step without further purification.

### Preparation of Compound N

Crude compound M, from the previous step, was taken into dioxane (5 mL) and cooled in an ice/water bath. An HCl solution in dioxane (4 N, 20 mL) was added. The mixture was stirred at ambient temperature for 3 h and then concentrated in vacuo to afford a yellowish foam. This material was dissolved in a mixture of DIEA (5.40 mL 31.2 mmol) in DMF (60 mL). mono-tButyl malonate (1.9 mL, 12.5 mmol) was added, followed by BOP (5.53 g, 12.5 mmol). The reaction mixture was stirred at ambient temperature for 14 h. The mixture was then concentrated and the residue was partitioned between EtOAc and water (60 mL/40 mL). The organic layer was removed and then washed with water (20 mL) and brine (20 mL), dried over Na₂SO₄ and concentrated. The residue was taken into THF (40 mL) at ambient temperature. TBAF (20.0 mL, 20.0 mmol, 1.0 M in THF) was added in one portion and the mixture was stirred for 4 h. The reaction mixture was then concentrated and the residue was partitioned between EtOAc and water (60 mL/40 mL). The organic layer was washed with water (2×20 mL), brine (20 mL), dried over Na₂SO₄ and concentrated. The residue was purified by silica gel column using a gradient of 1%-10% MeOH in EtOAc to afford compound N as a viscous oil (1.68 g, 1.67 mmol, 16% from compound M. LC-MS [M+H] 1006.4 (C₅₁H₇₁N₇O₁₂S+H, calc: 1006.5).

### Synthesis of N-methyl-N-(naloxone-6-enol-carbonyl-methyl amino)ethylamine-arginine-malonic acid (Compound AN-3)

Compound N (1.68 g, 1.67 mmol) was dissolved in a mixture of m-cresol (0.3 mL) in TFA (20 mL). The mixture was stirred at ambient temperature for 2 h. Most of the TFA was then removed in vacuo (˜95%). The residue was taken into MeOH (3 mL) and added dropwise to a stirred HCl solution in ether (20 mL, 2 M). The white solid was filtered and washed with ether (3×10 mL). The white solid was purified by prep HPLC, using a RP-18e C18 column (4.6×50 mm); flow rate 1.5 mL/min; mobile phase A: 0.1% TFA/water; mobile phase B 0.1% TFA/CH₃CN; gradient elution. Lyophilization of the collected fractions afforded the TFA salt of Compound AN-3, which was treated with aq. HCl (5 mL, 0.1 M) and lyophilized to give Compound AN-3, also referred to as Compound 3, as a white solid (490 mg, 38% yield, 99.4% purity by UV). LC-MS [M+H] 698.5 (C₃₄H₄₇N₇O₉+H, calc: 697.8).

### Example 3

### Synthesis of N-(naloxone-6-enol-carbonyl-methyl amino)ethylamine-D-arginine-malonic acid (Compound AN-4)

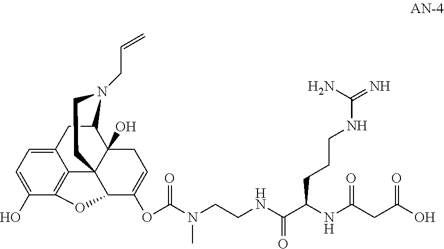

Compound AN-4 was prepared following the method described in Example 1 herein to prepare N-(naltrexone-6-enol-carbonyl-methyl amino)ethylamine-D-arginine-malonic acid (Compound AN-1), but using naloxone instead of naltrexone. LC-MS [M+H] 670.5 (C₃₂H₄₄N₇O₉+H, calc: 670.7).

### Example 4

### Synthesis of N-(naltrexone-6-enol-carbonyl-methyl amino)ethylamine-D-arginine-acetate (Compound AN-5)

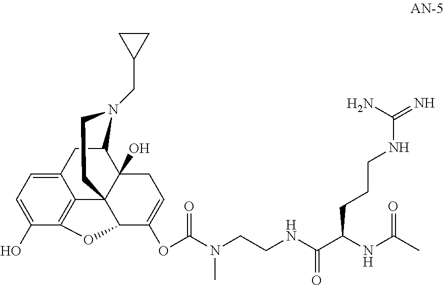

Compound AN-5 was prepared following the method described in Example 1 herein to prepare N-(naltrexone-6-enol-carbonyl-methyl amino)ethylamine-D-arginine-malonic acid (Compound AN-1), but using acetic anhydride instead of mono-tButyl malonate. LC-MS [M+H] 640.5 (C₃₂H₄₅N₇O₇+H, calc: 640.8).

### Example 5

### Preparation of N-(naltrexone-6-enol-carbonyl-methyl amino)ethylamine-D-lysine-malonic acid (Compound AN-6)

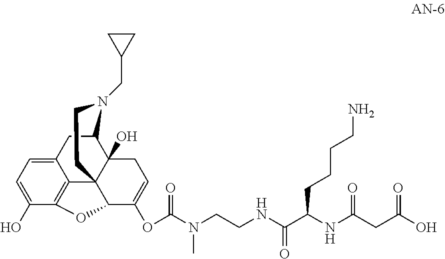

Compound AN-6 was prepared following the method described in Example 1 herein to prepare N-(naltrexone-6-enol-carbonyl-methyl amino)ethylamine-D-arginine-malonic acid (Compound AN-1), but using Fmoc-(D)-Lys(Boc)-OH instead of Boc-(D)-Arg(Pbf)-OH, piperidine for deprotection of the Fmoc group on α-nitrogen of Lys, and lastly TFA/DCM for Boc deprotection of the Lys reside. LC-MS [M+H] 656.5 (C₃₃H₄₅N₅O₉+H, calc: 656.8).

### Example 6

### Preparation of N-(naltrexone-6-enol-carbonyl-methyl amino)ethylamine-D-aspartic acid-acetate (Compound AN-7)

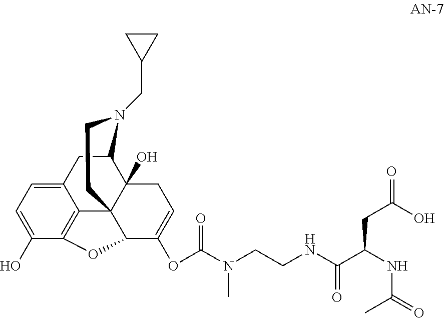

Compound AN-7 was prepared following the method described in Example 4 herein to prepare N-(naltrexone-6-enol-carbonyl-methyl amino)ethylamine-D-arginine-acetate (Compound AN-5), but using Fmoc-(D)-Asp(OtBu)-OH instead of Boc-(D)-Arg(Pbf)-OH, piperidine for deprotection of the Fmoc group on α-nitrogen of Asp, and lastly TFA/DCM for t-Bu deprotection of the Asp reside. LC-MS [M+H] 599.5 (C₃₀H₃₈N₄O₉+H, calc: 599.7).

### Example 7

### Preparation of N-(naltrexone-3-methyl ether-6-enol-carbonyl-methyl amino)ethylamine-D-arginine-malonic acid (Compound AN-8)

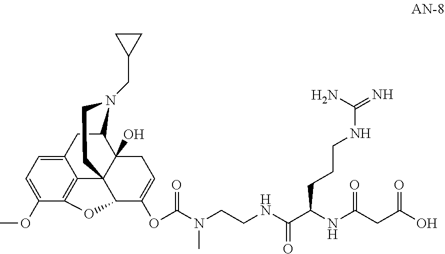

Compound AN-8 was prepared following the method described in Example 1 herein to prepare N-(naltrexone-6-enol-carbonyl-methyl amino)ethylamine-D-arginine-malonic acid (Compound AN-1), but using naltrexone 3-methyl ether instead of naltrexone. LC-MS [M+H] 698.5 (C₃₄H₄₇N₇O₉+H, calc: 698.8).

### Example 8

### Preparation of N-(hydromorphone-6-enol-carbonyl-methyl-amino)ethylamine-D-arginine-malonic acid (Compound AG-4)

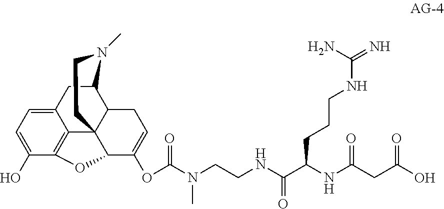

Compound AG-4 was prepared following the method described in Example 1 herein to prepare N-(naltrexone-6-enol-carbonyl-methyl amino)ethylamine-D-arginine-malonic acid (Compound AN-1), but using hydromorphone instead of naltrexone. LC-MS [M+H] 628.5 (C₃₄H₄₁N₇O₈+H, calc: 628.8).

### Biological Data

### Example 9

### In Vitro Human μ-Opioid Receptor Agonist and Antagonist Cellular Functional Assays

This Example measures the ability of certain compounds of the present disclosure to effect an agonist or antagonist response when exposed to recombinant human μ-opioid receptor expressed in CHO cells.

The general procedure follows the protocol described by Wang, J.-B., Johnson, P. S., Perscio, A. M., Hawkins, A. L., Griffin, C. A. and Uhl, G. R. (1994). FEBS Lett., 338: 217-222. More specifically, the assays included each of the compounds indicated in Table 1 and recombinant Chinese hamster ovary (CHO) cells expressing the human μ-opioid receptor on their cell surfaces. The control reaction included 1 μM DAMGO. The reaction mixtures were incubated at 37° C. for 10 min, and the reaction product was cyclic AMP (cAMP). The samples were submitted to homogeneous time resolved fluorescence (HTRF®). EC₅₀ values (concentration producing a half-maximal specific response) were determined by non-linear regression fit using the Hillplot software.

Table 1 provides agonist and antagonist EC₅₀ values for peripheral opioid antagonist Compound AN-1, also referred to as Compound 1 (which can be prepared as described in the Examples herein), peripheral opioid agonist Compound AG-2, also referred to as Compound 2 (which can be prepared as described in the Examples herein), naltrexone and oxycodone. Table 1 also provides the naltrexone-to-Compound AN-1 (NTX/Compound AN-1) and oxycodone-to-Compound AG-2 (OC/Compound AG-2) relative potencies (i.e., EC₅₀ at the human μ-opioid receptor) of naltrexone or oxycodone to Compound AN-1 and Compound AG-2, respectively.

TABLE 1
EC₅₀ values

| | | | NTX/Compound | OC/Compound |
|---|---|---|---|---|
| | Agonist | Antagonist | AN-1 relative | AG-2 relative |
| Compound | EC₅₀ | EC₅₀ | potency | potency |
| Naltrexone | | 3.0E−8 | | |
| Compound | | 1.8E−7 | 6.0 | |
| AN-1 | | | | |
| Oxycodone | 7.8E−8 | | | |
| Compound | 8.5E−7 | | | 10.9 |
| AG-2 | | | | |

The results in Table 1 show that peripheral opioid antagonist Compound AN-1 retains the ability to effect an antagonist response at the human μ-opioid receptor. In addition, peripheral opioid agonist Compound AG-2 retains the ability to effect an agonist response at the human μ-opioid receptor.

### Example 10

### Pharmacokinetics of Peripheral Opioid Antagonist Compound AN-1 Following PO Administration to Rats

This Example demonstrates the bioavailability and stability in plasma of peripheral opioid antagonist Compound AN-1 administered orally (PO) to rats.

Saline solutions of Compound AN-1 (which can be prepared as described in the Examples herein) were dosed as indicated in Table 2 via oral gavage into 4 jugular vein-cannulated male Sprague Dawley rats that had been fasted for 16-18 h prior to oral dosing. At specified time points, blood samples were drawn, harvested for plasma via centrifugation at 5,400 rpm at 4° C. for 5 min, and 100 microliters (μl) plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

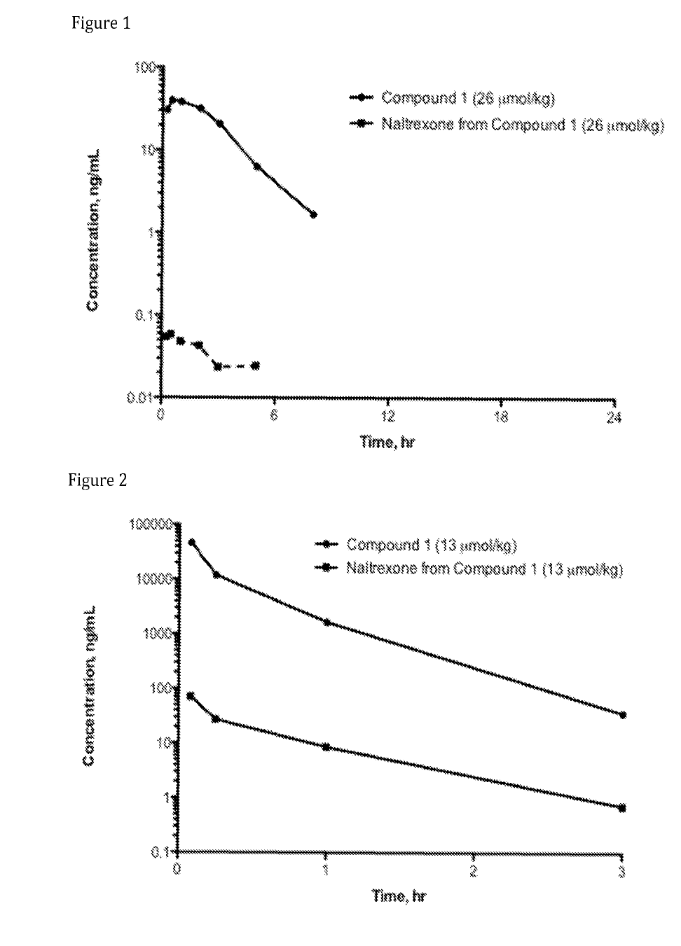

Table 2 and FIG. 1 provide Compound AN-1 and naltrexone exposure results for rats administered Compound AN-1 orally. Results in Table 2 are reported, for each group of rats, as (a) maximum plasma concentration (Cmax) of Compound AN-1 (average±standard deviation), (b) time after administration of Compound AN-1 to reach maximum Compound AN-1 concentration (Tmax) (average±standard deviation), (c) area under the curve (AUC) (average±standard deviation), from 0 to 8 h for Compound AN-1, and (d) maximum plasma concentration (Cmax) of naltrexone (NTX) released (average±standard deviation).

TABLE 2
Cmax, Tmax and AUC values of Compound AN-1 and
Cmax value of naltrexone in rat plasma

| Com- | Com- | Com- | Com- | | |
|---|---|---|---|---|---|
| pound | pound | pound | pound | | |
| AN-1 | AN-1 | AN-1 | AN-1 | Mean | NTX |
| Dose, | Dose, | Cmax ± sd, | Tmax ± sd, | AUC | Cmax ± sd, |
| mg/kg | μmol/kg | ng/mL | h | ng*h/mL | ng/mL |
| 20 | 26 | 41.0 ± 13* | 0.750 ± 0.29 | 139 ± 32 | 0.0666 ± 0.013\hat{ } |

*Lower limit of quantitation was 1.00 ng/mL
\hat{ }Lower limit of quantitation was 0.0250 ng/mL

FIG. 1 compares mean plasma concentrations over time of Compound AN-1 and of naltrexone released from Compound AN-1.

The results in Table 2 and FIG. 1 indicate that peripheral opioid antagonist Compound AN-1 is bioavailable and is stable when administered orally. For example, the plasma concentration of naltrexone released from Compound AN-1 is only about 0.16% of the plasma concentration of Compound AN-1.

### Example 11

### Pharmacokinetics of Peripheral Opioid Antagonist Compound AN-1 Following IV Administration to Rats

This Example compares the plasma concentrations of peripheral opioid antagonist Compound AN-1 and naltrexone in rats following intravenous (IV) administration of Compound AN-1.

Compound AN-1 (which can be prepared as described in the Examples herein) was dissolved in saline and injected into the tail vein of 4 jugular vein-cannulated male Sprague Dawley rats at a dose of 10 mg/kg. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by high performance liquid chromatography/mass spectrometry (HPLC/MS).

Table 3 and FIG. 2 provide Compound AN-1 and naltrexone exposure results for rats administered Compound AN-1 intravenously. Results in Table 3 are reported as maximum plasma concentration (Cmax) of Compound AN-1 and naltrexone (NTX), respectively, (average±standard deviation).

TABLE 3
Cmax values of Compound AN-1 and naltrexone in rat plasma

| Compound | Compound | | |
|---|---|---|---|
| AN-1 | AN-1 | | |
| Dose, | Dose, | Compound AN-1 | NTX Cmax ± sd, |
| mg/kg | μmol/kg | Cmax ± sd, ng/mL* | ng/mL\hat{ } |
| 10 | 13 | 46,300 ± 7,200 | 71.1 ± 13 |

*Lower limit of quantitation was 1.00 ng/mL
\hat{ }Lower limit of quantitation was 0.0250 ng/mL

Table 3 and FIG. 2 demonstrate that the plasma concentration of naltrexone in rats administered peripheral opioid antagonist Compound AN-1 intravenously is only 0.15% of the plasma concentration of Compound AN-1, indicating that IV administration of Compound AN-1 does not lead to significant release of naltrexone into plasma.

### Example 12

### Pharmacokinetics Following IV Administration of Peripheral Opioid Antagonist Compound AN-1 to Rats: Plasma and Cerebrospinal Fluid Penetration

This Example compares the plasma and cerebrospinal fluid (CSF) concentrations of peripheral opioid antagonist Compound AN-1 and naltrexone following intravenous (IV) administration of Compound AN-1 to rats. Plasma/CSF partitioning coefficients are predictive of the ability of a compound to penetrate the blood-brain barrier.

Compound AN-1 (which can be prepared as described in the Examples herein), at a dose of 10 mg/kg, was dissolved in saline and injected into the tail vein of 4 male Sprague Dawley rats. After 2 minutes, the rats were anesthetized by carbon dioxide asphyxiation and blood samples were drawn, harvested for plasma via centrifugation at 5,400 rpm at 4° C. for 5 min, and 100 μl plasma transferred from each sample into a fresh tube containing 2 μl of 50% formic acid. The CSF fluid was collected using a 22×1 inch gauge needle connected to polyurethane catheter type MRE-040 tubing (Braintree Scientific, Inc., Braintree, Mass.). The needle was inserted just below the nuchal crest at the area of the foramen magnum; clear CSF fluid was collected into the catheter and transferred into a collection tube. The CSF samples were centrifuged at 5,400 rpm at 4° C. for 5 min, and 100 μl CSF fluid transferred from each sample into a fresh tube. The plasma and CSF samples were immediately placed in dry ice and then stored in a −80° C. freezer until analysis by high performance liquid chromatography/mass spectrometry (HPLC/MS). In order to study Compound AN-1 and naltrexone plasma and CSF penetration over time, additional groups of 4 rats were administered Compound AN-1 as described above and anesthetized at specified time points. Plasma and CSF were collected and analyzed as described above. Results from these rats indicated that equilibrium was quickly reached in the plasma and CSF compartments after dosing and that the extent of partitioning between CSF and plasma was consistent across time points. Thus, only the 2-minute time point data are reported in Table 4.

Results in Table 4 are reported as mean concentrations of Compound AN-1 or of naltrexone released from Compound AN-1 in plasma or CSF. Table 4 also provides the plasma-to-CSF (plasma/CSF) partitioning coefficients, i.e., the ratios of concentration in the plasma to concentration in the CSF for Compound AN-1 and for naltrexone.

TABLE 4
Mean plasma and CSF concentration values and partitioning coefficients of
Compound AN-1 and naltrexone

| | Compound conc. | Compound conc. | Plasma/CSF partitioning |
|---|---|---|---|
| Compound | in plasma, ng/mL | in CSF, ng/mL | coefficient |
| Compound | 46,300 | 74.7 | 620 |
| AN-1 | | | |
| NTX from | 71.1 | 7.44 | 9.6 |
| Compound | | | |
| AN-1 | | | |

The results in Table 4 indicate that the relative plasma/CSF partitioning coefficient of peripheral opioid antagonist Compound AN-1 to naltrexone is about 65 (i.e., 620/9.6). Thus, Compound AN-1 is significantly less likely to cross the blood brain barrier than is naltrexone. The data reported herein demonstrate that peripheral opioid antagonist Compound AN-1 is a potent and stable peripheral opioid antagonist that is also bioavailable when administered orally.

### Example 13

### Pharmacokinetics of Peripheral Opioid Agonist Compound AG-2 Following PO Administration to Rats

This Example demonstrates the bioavailability and stability in plasma of peripheral opioid agonist Compound AG-2 administered orally (PO) to rats.

Saline solutions of Compound AG-2, also referred to Compound 2 (which can be prepared as described in the Examples herein) were dosed as indicated in Table 5 via oral gavage into 4 jugular vein-cannulated male Sprague Dawley rats that had been fasted for 16-18 h prior to oral dosing. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

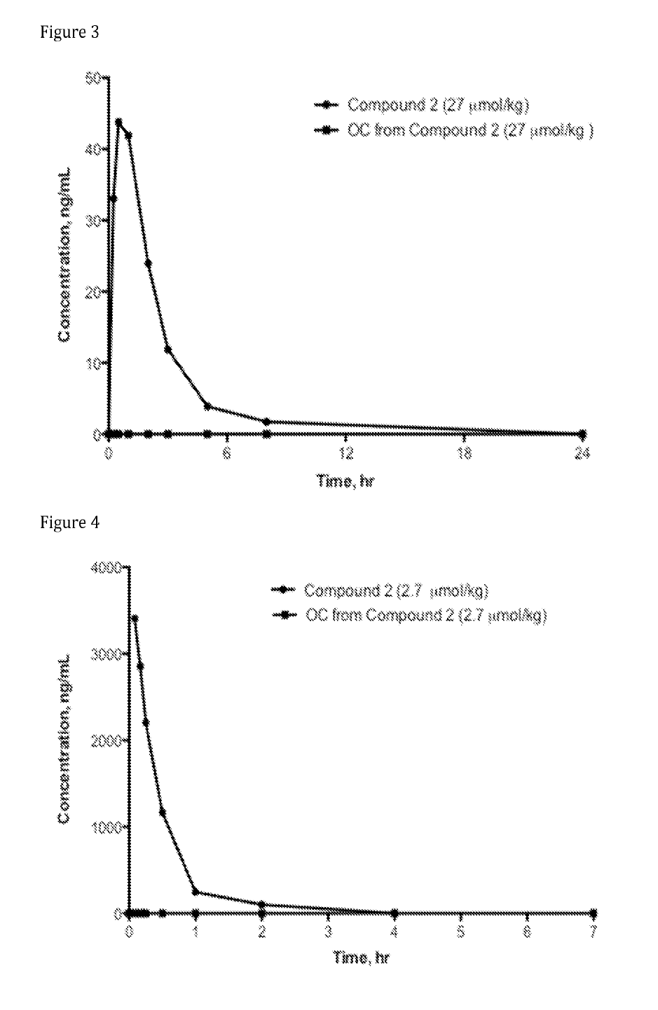

Table 5 and FIG. 3 provide Compound AG-2 and oxycodone exposure results for rats administered Compound AG-2 orally. Results in Table 5 are reported, for each group of rats, as (a) maximum plasma concentration (Cmax) of Compound AG-2 (average±standard deviation), (b) time after administration of Compound AG-2 to reach maximum Compound AG-2 concentration (Tmax) (average±standard deviation) and (c) maximum plasma concentration (Cmax) of oxycodone (OC) released (average±standard deviation).

TABLE 5
Cmax and Tmax values of Compound AG-2 and
Cmax value of oxycodone in rat plasma

| | | | Compound | Compound | |
|---|---|---|---|---|---|
| | | | AG-2 | AG-2 | OC |
| | Dose, | Dose, | Cmax ± sd, | Tmax ± sd, | Cmax ± sd, |
| Compound | mg/kg | μmol/kg | ng/mL | h | ng/mL |
| Compound | 20 | 27 | 46.1 ± 0.90* | 0.625 ± 0.25 | nc\hat{ } |
| AG-2 | | | | | |

*Lower limit of quantitation was 1.00 ng/mL
\hat{ }Lower limit of quantitation (LLOQ) was 0.100 ng/mL
nc = not calculable below LLOQ

FIG. 3 compares mean plasma concentrations over time of Compound AG-2 and of oxycodone released from Compound AG-2.

The results in Table 5 and FIG. 3 indicate that peripheral opioid agonist Compound AG-2 is bioavailable and is stable when administered orally. For example, the plasma concentration of oxycodone release from Compound AG-2 is undetectable when measured in an assay having a lower limit of quantitation of 0.100 ng/ml.

### Example 14

### Pharmacokinetics of Peripheral Opioid Agonist Compound AG-2 Following IV Administration to Rats

This Example compares the plasma concentrations of peripheral opioid agonist Compound AG-2 and oxycodone in rats following intravenous (IV) administration of Compound AG-2.

Compound AG-2 (which can be prepared as described in the Examples herein) was dissolved in saline and injected into the tail vein of 4 jugular vein-cannulated male Sprague Dawley rats at a dose of 2 mg/kg. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by high performance liquid chromatography/mass spectrometry (HPLC/MS).

Table 6 and FIG. 4 provide Compound AG-2 and oxycodone exposure results for rats administered Compound AG-2 intravenously. Results in Table 6 are reported as maximum plasma concentration (Cmax) of Compound AG-2 and oxycodone (OC), respectively, (average±standard deviation).

TABLE 6
Cmax values of Compound AG-2 and oxycodone in rat plasma

| Compound | Compound | | |
|---|---|---|---|
| AG-2 | AG-2 | | |
| Dose, | Dose, | Compound AG-2 | OC Cmax ± sd, |
| mg/kg | μmol/kg | Cmax ± sd, ng/mL* | ng/mL\hat{ } |
| 2 | 2.7 | 3,410 ± 130 | nc |

*Lower limit of quantitation was 1.00 ng/mL
\hat{ }Lower limit of quantitation (LLOC) was 0.100 ng/mL
nc = not calculable below LLOQ

Table 6 and FIG. 4 demonstrate that the plasma concentration of oxycodone in rats administered peripheral opioid agonist Compound AG-2 intravenously is undetectable when measured in an assay having a lower limit of quantitation of 0.100 ng/ml, indicating that IV administration of Compound AG-2 does not lead to significant release of oxycodone into plasma.

### Example 15

### Pharmacokinetics Following IV Administration of Peripheral Opioid Agonist Compound AG-2 to Rats: Plasma and Cerebrospinal Fluid Penetration

This Example compares the plasma and cerebrospinal fluid (CSF) concentrations of peripheral opioid agonist Compound AG-2 and oxycodone following intravenous (IV) administration of the respective compounds to rats. Plasma/CSF partitioning coefficients are predictive of the ability of a compound to penetrate the blood-brain barrier.

Compound AG-2 (which can be prepared as described in the Examples herein), at a dose of 10 mg/kg, or an equimole dose of oxycodone, each was dissolved in saline and injected into the tail vein of 4 male Sprague Dawley rats. After 2 minutes, the rats were anesthetized by carbon dioxide asphyxiation and blood samples were drawn. Plasma and CSF were collected and analyzed as described in Example 12 herein. In order to study Compound AG-2 and oxycodone plasma and CSF penetration over time, additional groups of 4 rats were administered Compound AG-2 or oxycodone as described above and anesthetized at specified time points. Plasma and CSF were collected and analyzed as described above. Results from these rats indicated that equilibrium was quickly reached in the plasma and CSF compartments after dosing and that the extent of partitioning between CSF and plasma was consistent across time points. Thus, only the 2-minute time point data are reported in Table 7.

Results in Table 7 are reported, for each group of 4 rats, as mean concentration of the indicated compounds in plasma or CSF. Table 7 also provides the plasma-to-CSF (plasma/CSF) partitioning coefficients, i.e., the ratios of concentration in the plasma to concentration in the CSF of the indicated compounds.

TABLE 7
Mean plasma and CSF concentration values and partitioning coefficients of
Compound AG-2 and oxycodone

| | | | Plasma/CSF |
|---|---|---|---|
| | Compound conc. | Compound conc. | partitioning |
| Compound | in plasma, ng/mL | in CSF, ng/mL | coefficient |
| Compound | 28,600 | 22.8 | 1,254 |
| AG-2 | | | |
| OC | 10,300 | 2,158 | 4.8 |

The results in Table 7 indicate that the relative plasma/CSF partitioning coefficient of peripheral opioid agonist Compound AG-2 to oxycodone is about 263 (i.e., 1,254/4.8). Thus, Compound AG-2 is significantly less likely to cross the blood brain barrier than is oxycodone. The data reported herein demonstrate that peripheral opioid agonist Compound AG-2 is a potent and stable peripheral opioid agonist that is also bioavailable when administered orally.

### Example 16

### Pharmacokinetics of Peripheral Opioid Antagonist Compound AN-3 Following PO Administration to Rats

This Example demonstrates the bioavailability and stability in plasma of peripheral opioid antagonist Compound AN-3 administered orally (PO) to rats.

Saline solutions of Compound AN-3, also known as Compound 3 (which can be prepared as described in the Examples herein) were dosed as indicated in Table 8 via oral gavage into 4 jugular vein-cannulated male Sprague Dawley rats that had been fasted for 16-18 h prior to oral dosing. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

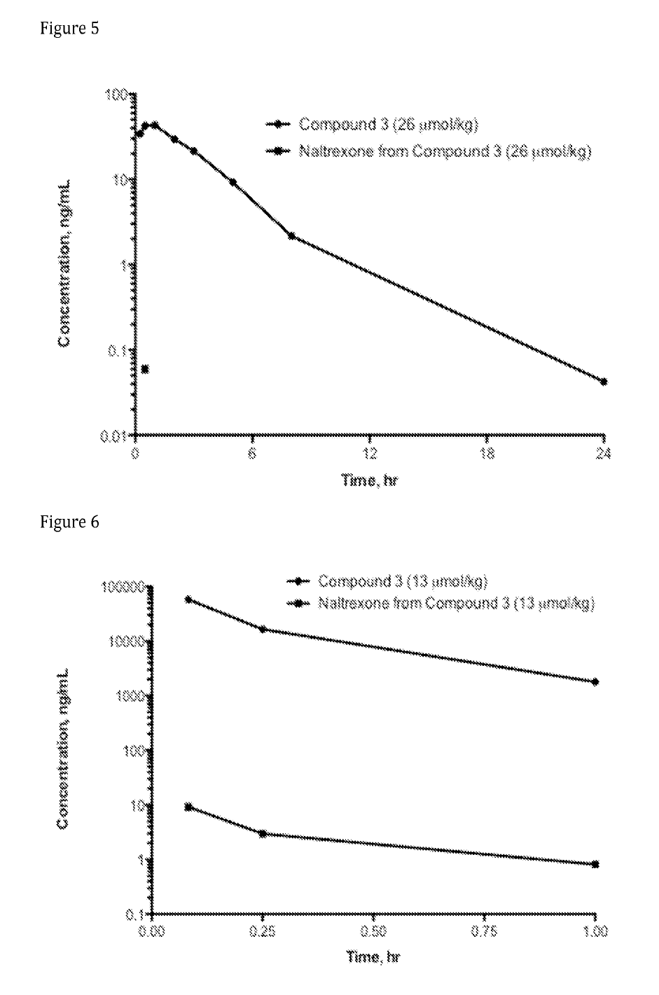

Table 8 and FIG. 5 provide Compound AN-3 naltrexone exposure results for rats administered Compound AN-3 orally. Results in Table 8 are reported, for each group of rats, as (a) maximum plasma concentration (Cmax) of Compound AN-3 (average±standard deviation), (b) time after administration of Compound AN-3 to reach maximum Compound AN-3 concentration (Tmax) (average±standard deviation) and (c) maximum plasma concentration (Cmax) of naltrexone (NTX) (average±standard deviation).

TABLE 8
Cmax, Tmax and AUC values of Compound AN-3 and
Cmax value of naltrexone in rat plasma

| Compound | Compound | Compound | Compound | |
|---|---|---|---|---|
| AN-3 | AN-3 | AN-3 | AN-3 | |
| Dose, | Dose, | Cmax ± | Tmax ± sd, | NTX Cmax ± sd, |
| mg/kg | μmol/kg | sd, ng/mL | h | ng/mL |
| 20 | 26 | 45.5 ± 13* | 0.750 ± 0.29 | 0.0596 ± 0.083\hat{ } |

*Lower limit of quantitation was 0.100 ng/mL
\hat{ }Lower limit of quantitation (LLOQ) was 0.0500 ng/mL

FIG. 5 compares mean plasma concentrations over time of Compound AN-3 and of naltrexone released from Compound AN-3. There is only one time point shown as naltrexone concentrations at other time points were below the LLOQ.

The results in Table 8 and FIG. 5 indicate that peripheral opioid antagonist Compound AN-3 is bioavailable and is stable when administered orally. For example, the plasma concentration of naltrexone released from Compound AN-3 is only about 0.13% of the plasma concentration of Compound AN-3.

### Example 17

### Pharmacokinetics of Peripheral Opioid Antagonist Compound AN-3 Following IV Administration to Rats

This Example compares the plasma concentrations of peripheral opioid antagonist Compound AN-3 and naltrexone in rats following intravenous (IV) administration of Compound AN-3.

Compound AN-3 (which can be prepared as described in the Examples herein) was dissolved in saline and injected into the tail vein of 4 jugular vein-cannulated male Sprague Dawley rats at a dose of 10 mg/kg. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by high performance liquid chromatography/mass spectrometry (HPLC/MS).

Table 9 and FIG. 6 provide Compound AN-3 and naltrexone exposure results for rats administered Compound AN-3 intravenously. Results in Table 9 are reported as maximum plasma concentration (Cmax) of Compound AN-3 and naltrexone (NTX), respectively, (average±standard deviation).

TABLE 9
Cmax values of Compound AN-3 and naltrexone in rat plasma

| Compound | Compound | | |
|---|---|---|---|
| AN-3 | AN-3 | | |
| Dose, | Dose, | Compound AN-3 | NTX Cmax ± sd, |
| mg/kg | μmol/kg | Cmax ± sd, ng/mL* | ng/mL\hat{ } |
| 10 | 13 | 57,900 ± 4,100 | 9.17 ± 0.85 |

*Lower limit of quantitation was 100 ng/mL
\hat{ }Lower limit of quantitation was 0.0250 ng/mL

Table 9 and FIG. 6 demonstrate that the plasma concentration of naltrexone in rats administered peripheral opioid antagonist Compound AN-3 intravenously is only 0.015% of the plasma concentration of Compound AN-3, indicating that IV administration of Compound AN-3 does not lead to significant release of naltrexone into plasma.

### Example 18

### Pharmacokinetics Following IV Administration of Peripheral Opioid Antagonist Compound AN-3 to Rats: Plasma and Cerebrospinal Fluid Penetration

This Example compares the plasma and cerebrospinal fluid (CSF) concentrations of peripheral opioid antagonist Compound AN-3 and naltrexone following intravenous (IV) administration of Compound AN-3 to rats. Plasma/CSF partitioning coefficients are predictive of the ability of a compound to penetrate the blood-brain barrier.

Compound AN-3 (which can be prepared as described in the Examples herein), at a dose of 10 mg/kg, was dissolved in saline and injected into the tail vein of 4 male Sprague Dawley rats. After 2 minutes, the rats were anesthetized by carbon dioxide asphyxiation and blood samples were drawn. Plasma and CSF were collected and analyzed as described in Example 12 herein. In order to study Compound AN-3 and naltrexone plasma and CSF penetration over time, additional groups of 4 rats were administered Compound AN-3 as described above and anesthetized at specified time points. Plasma and CSF were collected and analyzed as described above. Results from these rats indicated that equilibrium was quickly reached in the plasma and CSF compartments after dosing and that the extent of partitioning between CSF and plasma was consistent across time points. Thus, only the 2-minute time point data are reported in Table 10.

Results in Table 10 are reported as mean concentrations of Compound AN-3 or of naltrexone released from Compound AN-3 in plasma or CSF. Table 10 also provides the plasma-to-CSF (plasma/CSF) partitioning coefficients, i.e., the ratios of concentration in the plasma to concentration in the CSF for Compound AN-3 and for naltrexone.

TABLE 10
Mean plasma and CSF concentration values and partitioning coefficients of
Compound AN-3 and naltrexone

| | Compound conc. | Compound conc. | Plasma/CSF partitioning |
|---|---|---|---|
| Compound | in plasma, ng/mL | in CSF, ng/mL | coefficient |
| Compound | 57,900 | 74.4 | 778 |
| AN-3 | | | |
| NTX from | 9.17 | 0.813 | 11.3 |
| Compound | | | |
| AN-3 | | | |

The results in Table 10 indicate that the relative plasma/CSF partitioning coefficient of peripheral opioid antagonist Compound AN-3 to naltrexone is about 69 (i.e., 778/11.3). Thus, Compound AN-3 is significantly less likely to cross the blood brain barrier than is naltrexone.

### Example 19

### Pharmacokinetics of Peripheral Opioid Agonist Compound AG-4 Following PO Administration to Rats

This Example demonstrates the bioavailability and stability in plasma of peripheral opioid agonist Compound AG-4 administered orally (PO) to rats.

Saline solutions of Compound AG-4 (which can be prepared as described in the Examples herein) were dosed as indicated in Table 11 via oral gavage into 4 jugular vein-cannulated male Sprague Dawley rats that had been fasted for 16-18 h prior to oral dosing. At specified time points, blood samples were drawn, harvested for plasma via centrifugation at 5,400 rpm at 4° C. for 5 min, and 100 μl plasma transferred from each sample into a fresh tube containing 2 μl of 50% formic acid. The tubes were vortexed for 5-10 seconds, immediately placed in dry ice and then stored in a −80° C. freezer until analysis by HPLC/MS.

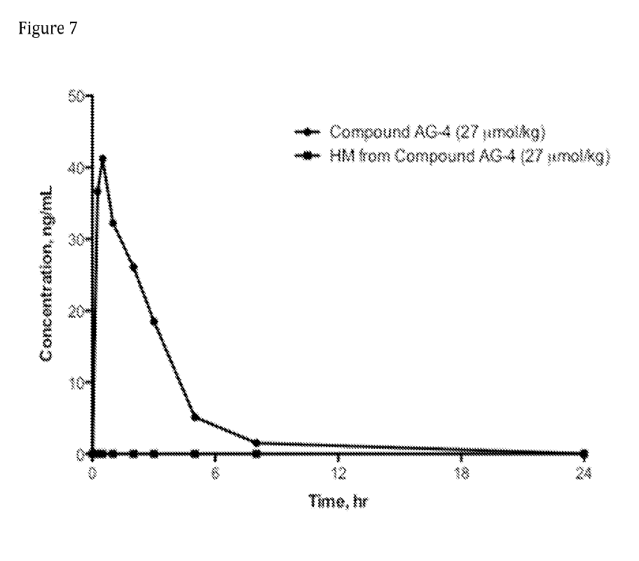

Table 11 and FIG. 7 provide Compound AG-4 and hydromorphone exposure results for rats administered Compound AG-4 orally. Results in Table 11 are reported, for each group of rats, as (a) maximum plasma concentration (Cmax) of Compound AG-4 (average±standard deviation), (b) time after administration of Compound AG-4 to reach maximum Compound AG-4 concentration (Tmax) (average±standard deviation) and (c) maximum plasma concentration (Cmax) of hydromorphone (HM) released from Compound AG-4 (average±standard deviation).

TABLE 11
Cmax and Tmax values of Compound AG-4 and
Cmax value of hydromorphone in rat plasma

| | | | Compound 2 | | |
|---|---|---|---|---|---|
| | Dose, | Dose | Cmax ± sd, | Compound 2 | HM Cmax ± |
| Compound | mg/kg | μmol/kg | ng/mL | Tmax ± sd, h | sd, ng/mL |
| Compound | 19 | 27 | 43.2 ± 7.1 * | 0.375 ± 0.14 | nc \hat{ } |
| AG-4 | | | | | |

* Lower limit of quantitation was 0.100 ng/mL
\hat{ } Lower limit of quantitation (LLOQ) was 0.050 ng/mL
nc = not calculable below LLOQ

FIG. 7 compares mean plasma concentrations over time of Compound AG-4 and of hydromorphone released from Compound AG-4.

The results in Table 11 and FIG. 7 indicate that peripheral opioid agonist Compound AG-4 is bioavailable and is stable when administered orally. For example, the plasma concentration of hydromorphone release from Compound AG-4 is undetectable when measured in an assay having a lower limit of quantitation of 0.050 ng/ml.

### Example 20

### Pharmacokinetics Following IV Administration of Peripheral Opioid Antagonist Compounds of the Embodiments to Rats: Plasma and Cerebrospinal Fluid Penetration

This Example demonstrates plasma/CSF partitioning coefficients of peripheral opioid antagonists of the embodiments following intravenous (IV) administration of such compounds to rats. Plasma/CSF partitioning coefficients are predictive of the ability of a compound to penetrate the blood-brain barrier.

Compound AN-4, Compound AN-6, and Compound AN-7 (which can be prepared as described in the Examples herein), each at a dose of 10 mg/kg, was dissolved in saline and injected into the tail vein of 4 male Sprague Dawley rats, using methods as described in Example 12 herein. Samples were collected and stored as described in Example 12 herein. The 2-min time point data are reported in Table 12.

Results in Table 12 are reported as mean concentrations of Compound AN-4, Compound AN-6, and Compound AN-7 in plasma or CSF. Table 12 also provides plasma-to-CSF (plasma/CSF) partitioning coefficients, i.e., the ratios of concentration in the plasma to concentration in the CSF for the respective compounds.

TABLE 12
Mean plasma and CSF concentration values and partitioning coefficients of
Compound AN-4, Compound AN-6, and Compound AN-7

| | Compound | | Plasma/CSF |
|---|---|---|---|
| | conc. in | Compound conc. in | partitioning |
| Compound | plasma, ng/mL | CSF, ng/mL | coefficient |
| Compound AN-4 | 49,200 | 101 | 487 |
| Compound AN-6 | 79,400 | 95.0 | 836 |
| Compound AN-7 | 76,600 | 462 | 166 |

The results in Table 12, as well as the results disclosed in other examples herein, indicate that peripheral opioid compounds of the embodiments are peripherally-restricted; i.e., compounds of the embodiments are significantly less likely to cross the blood brain bather than are the respective unmodified opioid drugs (e.g., oxycodone, naloxone, naltrexone).

### Example 21

### Effect of Peripheral Opioid Antagonists of the Embodiments on Morphine Induced Analgesia in Conscious Rats

This Example demonstrates the effect of peripheral opioid antagonists on CNS opioid receptors.

A rat tail flick latency assay (see, e.g., Tejwani G et al., 2002, Anesth Analg 94, 1542-1546) was used to determine whether peripheral opioid antagonists of the embodiments were capable of effecting antagonist activity at opioid receptors in the central nervous system. In such an assay, the tails of rats are exposed to a noxious stimulus, such as heat, and tail withdrawal latency is measured. The latency (i.e., time between exposure and reaction) is an indication of the amount of pain experienced by the rat. Rats pre-treated with morphine, an analgesic agent that exerts its effect at CNS opioid receptors, exhibit a long latency period. If such morphine-pre-treated rats are exposed to a CNS-penetrant opioid antagonist, such as naltrexone, the latency period is shortened due to antagonist inhibition of the analgesic effect. In contrast, exposure to a non-CNS-penetrant opioid antagonist (i.e., a peripheral opioid antagonist) would have no effect on the latency period in such an assay.

In this study, the tails of healthy male Sprague Dawley rats (5 per group) were painted with India ink (i.e., a 6-cm portion, starting at the tip). The rats were then placed in Broome Restrainers and allowed to acclimate for at least 15 min. At the conclusion of the acclimation period, baseline tail withdrawal latencies (baseline latency) were measured by exposing the rats to a focused light beam of 80 volts at a position 6.5 cm above the tail until the rats flicked their tail out of the path of the light beam. This step was repeated twice for each rat, in two spots, i.e., 2 cm and 4 cm from the tip of the tail. The responses were averaged and used as a baseline latency value for comparison to post-drug treatment latency values. In an effort to reduce tissue damage caused by repeated administration of the light beam, a maximum cut-off time of 15 sec was utilized; if an animal did not respond to the light beam within 15 sec, the beam was shut off, and a score of 15 was recorded for that time point.

Once baseline latency values were calculated, the rats were administered 5 mg/kg morphine at a volume of 1 ml/kg subcutaneously, and returned to their respective restrainers. The tail flick latency responses were measured at specific time points post morphine administration.

To determine the effect of opioid antagonism on rats pre-treated with morphine, the rats, 70 min after the morphine dose, were administered subcutaneously peripheral opioid antagonist Compound AN-1 or Compound AN-6 of the embodiments (which can be prepared as described in the Examples herein) or naltrexone (a CNS-penetrant opioid antagonist) as indicated in Table 13; each antagonist was formulated in saline at a volume of 1 ml/kg, The tail flick latency responses were measured at specific time points post compound administration (post-drug latency).

Table 13 indicates the dose amounts for each group of 5 rats administered morphine with or without a subsequent dose of Compound AN-1, Compound AN-6, or naltrexone.

TABLE 13
Drug amount(s) per rat

| | Morphine | Compound | Compound Dose, |
|---|---|---|---|
| Drug | Dose, mg/kg | dose, mg/kg | μmol/kg |
| Morphine alone | 5 | 0 | 0 |
| Morphine/Naltrexone | 5 | 1.8 | 4.8 |
| Morphine/Compound AN-1 | 5 | 0.3 | 0.4 |
| Morphine/Compound AN-6 | 5 | 0.3 | 0.4 |

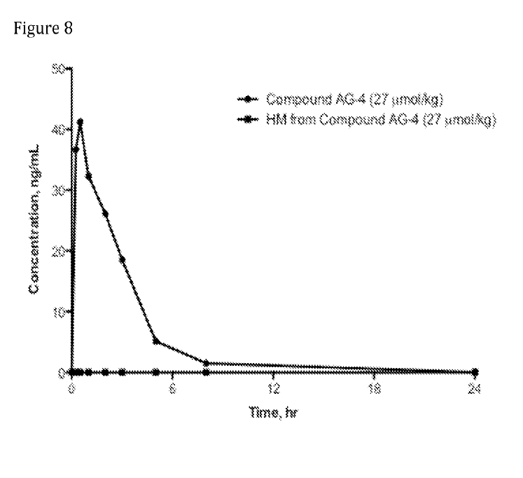

FIG. 8 provides percent maximal possible effect (% MPE) over time in a tail flick assay of Compound AN-1, Compound AN-6, and naltrexone administered subcutaneously to rats pre-treated with morphine; % MPE=[post-drug latency−baseline latency)/(cut-off time−baseline latency)]×100%.

The results in FIG. 8 indicate that animals dosed with morphine rapidly achieved and sustained analgesia. Rats subsequently dosed with peripheral opioid antagonists Compound AN-1 and Compound AN-6 showed no inhibition of analgesia whereas rats dosed with naltrexone show rapid inhibition of analgesia. Thus, peripheral opioid antagonists of the embodiments do not effect antagonism at central opioid receptors.

### Example 22

### Gastrointestinal Transit in Rats Following Oral Administration of Peripheral Opioid Antagonists of the Embodiments

This Example demonstrates peripheral antagonist effects of peripheral opioid antagonists of the embodiments.

Although opioid agonists are useful in the treatment of pain, they cause side effects when they interact with peripheral opioid receptors, for example, in the gastrointestinal (GI) tract. The most common of these side effects is constipation, which is mediated via peripheral receptors. A well-established model to assess the ability of peripheral opioid antagonists to inhibit the peripheral actions of opioid agonists is the rat gastrointestinal transit (GI transit) assay using activated charcoal as a marker (see, e.g., Manara L et al., 1986, J Pharmacol Exp Ther 237, 945-949). GI transit is expressed as the distance traveled by the charcoal suspension as a percentage of the total length of the small intestine. An opioid agonist, such as hydromorphone, inhibits movement of the charcoal suspension through the small intestine (i.e., decreased GI transit). In contrast, a peripheral opioid antagonist given in conjunction with the opioid agonist will inhibit the opioid agonist activity, leading to increased movement of the charcoal suspension through the small intestine (i.e., increased GI transit).

Sprague-Dawley rats (5 per group), fasted for approximately 18 to 20 hours prior to dose administration, were used for this study. Groups of rats received a single dose via oral gavage of 45 mg/kg hydromorphone HCl immediately followed by oral gavage of a saline solution or a compound of the embodiments (which can be prepared as described in the Examples herein) as indicated in Table 14; the compounds were dissolved in a saline solution at a dose volume of 2 mL/kg. A control group received saline only. One hour after such administrations, the rats were administered a charcoal suspension [10% (w/v) activated charcoal powder (Sigma-Aldrich, St. Louis, Mo.) and 2.5% (w/v) gum arabic (Sigma-Aldrich, St. Louis, Mo.) in deionized water] via oral gavage as a single dose at a volume of 10 mL/kg. Twenty minutes after administration of the charcoal suspension, the rats were euthanized, and the GI transit was determined. Assessment of GI transit was based on the measurement of movement of the charcoal suspension through the small intestine (percentage of the small intestine traversed by the leading edge of the charcoal transit marker) using the following equation: % transit=(C/SI)×100, where C was the distance traveled by the charcoal (mm) and SI was the total length of the small intestine (mm).

Table 14 indicates the dosing for each group of 5 rats for Compound AN-1, Compound AN-4, Compound AN-5, Compound AN-6, and Compound AN-8.

TABLE 14
Drug amount(s) per rat

| | | Compound | Compound |
|---|---|---|---|
| | Hydromorphone | Dose, | Dose, |
| Drug(s) | Dose, mg/kg | mg/kg | μmol/kg |
| Hydromorphone (HM) | 45 | 0 | 0 |
| HM/Compound AN-1 | 45 | 100 | 130 |
| HM/Compound AN-1 | 45 | 200 | 260 |
| HM/Compound AN-1 | 45 | 400 | 530 |
| HM/Compound AN-4 | 45 | 100 | 130 |
| HM/Compound AN-5 | 45 | 100 | 140 |
| HM/Compound AN-6 | 45 | 100 | 140 |
| HM/Compound AN-8 | 45 | 100 | 130 |

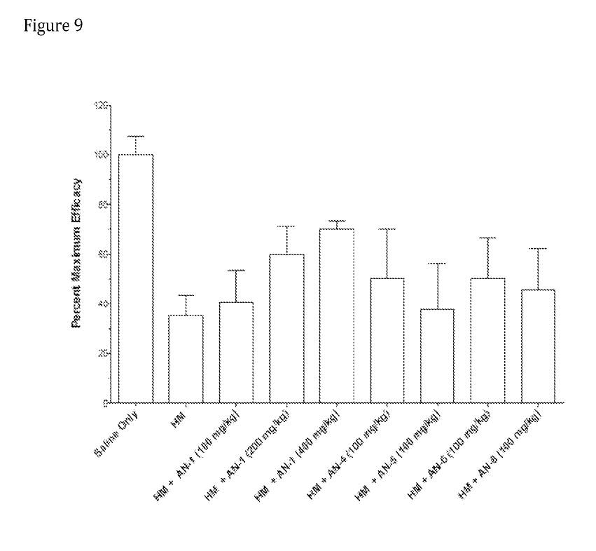

FIG. 9 provides percent maximum efficacy of GI transit for rats administered hydromorphone alone, hydromorphone with peripheral opioid antagonists of the embodiments, or saline, wherein GI transit for saline is assumed to provide 100% maximum efficacy.

The results in FIG. 9 indicate that the peripheral opioid antagonists of the embodiments are bioavailable when administered orally to rats and are able inhibit the effect of hydromorphone on GI transit, resulting in increased GI transit compared to oral administration of hydromorphone alone.

### Example 23

### Effect of Peripheral Opioid Agonist Compound AG-2 on Inflammatory Pain in Rats

This Example demonstrates the effect of oral administration of a peripheral opioid agonist of the embodiments on inflammatory pain in rats.

The carrageenan-induced inflammatory paw model (see, e.g., Bileviciute-Ljungar I et al, 2006, J Pharmacol Exp Ther 317, 220-227) was used to assess the ability of peripheral opioid agonist Compound AG-2 to reduce inflammatory pain, by measuring paw withdrawal latency in response to mechanical stimulation of the paw.

Sprague-Dawley rats, fasted for approximately 18 hours prior to compound administration, were used for this study. Rats were injected in the plantar surface of the right hindpaw with 100 ul of a 1% carrageenan solution formulated in water (carrageenan available from Spectrum Chemical Manufacturing Corporation, Gardena, Calif.) three hours prior to compound administration, in order to elicit maximum edema formation. The hindpaw withdrawal latency to mechanical stimulation for each paw was tested before compound administration for use as a pre-treatment baseline, and at specific time points after compound administration using the Randall Selitto test (instrument available from IITC Inc. Life Science, Woodland Hills, Calif.). Briefly, the hindpaw was pinched until the animal retracted its paw. The amount of force (in grams) required to elicit a withdrawal response was assessed. Two repeat measures were taken at each time point, and the averaged value from the inflamed paw was used to quantify the percentage of change from the pre-treatment baseline. In this study, 200 mg/kg of Compound AG-2 (which can be prepared as described in the Examples herein, and formulated in water at a concentration of 100 mg/ml) was administered to a group of 6 rats via oral gavage three hours after carrageenan administration. Another group of 12 rats received no compound (untreated).

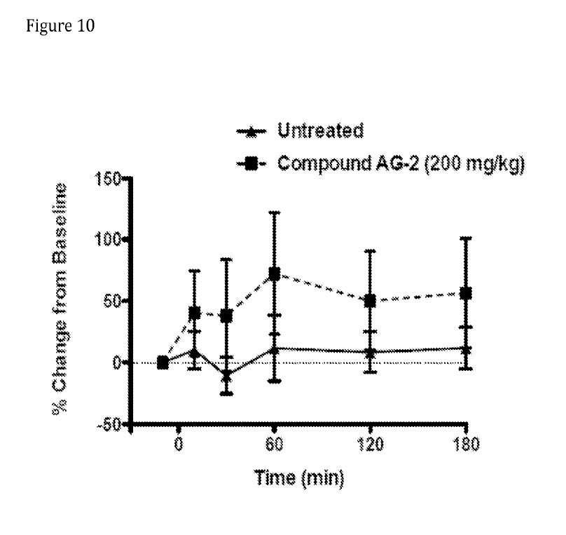

FIG. 10 provides the percent change from baseline over time in a carrageenan-induced inflammatory paw model of rats administered peripheral opioid agonist Compound AG-2 and or no compound.

The results in FIG. 10 indicate that rats dosed orally with peripheral opioid agonist Compound AG-2 had an increase in pain tolerance of approximately 50% compared to the untreated rats. This anti-inflammatory effect was sustained throughout the duration of the study. These results indicate that peripheral opioid agonists of the embodiments can reduce inflammatory pain.

### Example 24

### In Vitro Human μ-Opioid Receptor Antagonist and Agonist Cellular Functional Assays

This Example measures the ability of certain compounds of the present disclosure to effect an agonist or antagonist response when exposed to recombinant human μ-opioid receptor expressed in CHO cells.

Each of the compounds indicated in Table 15 was assayed and analyzed using methods similar to those described in Example 9 herein.

Table 15 provides agonist and antagonist EC₅₀ values for peripheral opioid antagonist Compound AN-7 (which can be prepared as described in the Examples herein), peripheral opioid agonist Compound AG-4 (which can be prepared as described in the Examples herein), naltrexone and hydromorphone. Table 15 also provides the naltrexone-to-Compound AN-7 (NTX/Compound AN-7) and hydromorphone-to-Compound AG-4 (HM/Compound AG-4) relative potencies (i.e., EC₅₀ at the human μ-opioid receptor) of naltrexone or hydromorphone to Compound AN-7 and Compound AG-4, respectively.

TABLE 15
EC₅₀ values

| | | | HM/Compound | NTX/Compound |
|---|---|---|---|---|
| | Agonist | Antagonist | AG-4 relative | AN-7 relative |
| Compound | EC₅₀ | EC₅₀ | potency | potency |
| Compound AN-7 | | 3.0E−07 | | 8.6 |
| Naltrexone | | 3.5E−08 | | |
| Compound AG-4 | 3.7E−08 | | 16.1 | |
| Hydromorphone | 2.3E−09 | | | |

The results in Table 15 show that peripheral opioid antagonist Compound AN-7 retains the ability to effect a potent antagonist response at the human μ-opioid receptor. In addition, peripheral opioid agonist Compound AG-4 retains the ability to effect a potent agonist response at the human μ-opioid receptor.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of formula (I): 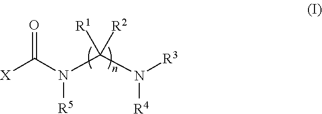
wherein:
X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 2 to 10;
R³ is selected from hydrogen, alkyl, substituted alkyl, aryl, and substituted aryl;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or R⁴ is 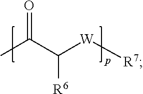
each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;
each W is independently —NR⁸—;
each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
p is an integer from one to five; and
R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and
provided that:
1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is 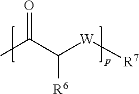
or a salt, hydrate or solvate thereof.

2. A compound according to claim 1, of formula (II): 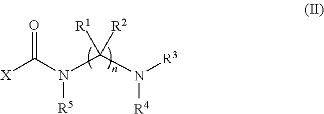
wherein:
X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 2 to 10;
R³ is selected from alkyl, substituted alkyl, aryl, and substituted aryl;
R⁴ is 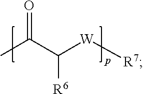
each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;
each W is independently —NR⁸—;
each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
p is an integer from one to five; and
R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol;
or a salt, hydrate or solvate thereof.

3. A compound according to claim 1, of formula (III): 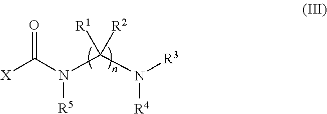
wherein:
X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 2 to 10;
R³ is selected from alkyl, substituted alkyl, aryl, and substituted aryl;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or a salt, hydrate or solvate thereof.

4. A compound according to claim 1, of formula (IV): 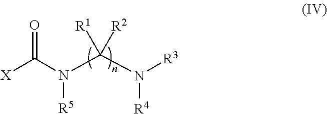
wherein:
X represents a residue of a ketone-containing opioid, wherein the hydrogen atom of the corresponding enolic group or reduced enolic group of the ketone is replaced by a covalent bond to —C(O)—NR⁵—(C(R¹)(R²))ₙ—NR³R⁴; or wherein the hydrogen atom of an amino group that is generated from reductive amination of the ketone of the ketone-containing opioid is replaced by a covalent bond to —C(O)—NR⁵—(C(R¹)(R²))ₙ—NR³R⁴;
R⁵ is selected from hydrogen, alkyl, substituted alkyl, arylalkyl, substituted arylalkyl, aryl and substituted aryl;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached form a cycloalkyl, substituted cycloalkyl, aryl, or substituted aryl group, or two R² or R³ groups on adjacent carbon atoms, together with the carbon atoms to which they are attached, form a cycloalkyl, substituted cycloalkyl, aryl, or substituted aryl group;
n is an integer from 2 to 10;
R³ is hydrogen;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or a salt, hydrate or solvate thereof.

5. A compound of formula (V): 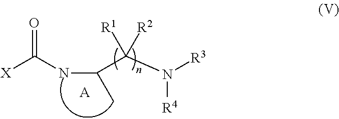
wherein:
X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;
the A ring is a heterocyclic 5 to 12-membered ring;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;
n is an integer from 1 to 10;
R³ is selected from hydrogen, alkyl, substituted alkyl, aryl, and substituted aryl;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or R⁴ is 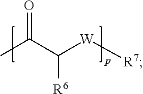
each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;
each W is independently —NR⁸—;
each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
p is an integer from one to five; and
R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol; and
provided that:
1) when R³ is hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
2) when R³ is not hydrogen, then R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid; or R⁴ is 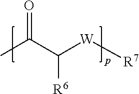
or a salt, hydrate or solvate thereof.

6. A compound according to claim 5, of formula (VI): 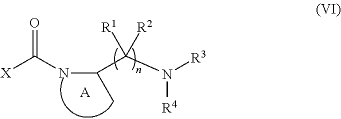
wherein:
X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;
the A ring is a heterocyclic 5 to 12-membered ring;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;
n is an integer from 1 to 10;
R³ is selected from alkyl, substituted alkyl, aryl, and substituted aryl;
R⁴ is 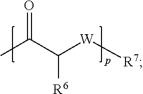
each R⁶ is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or optionally, R⁶ and R⁷ together with the atoms to which they are bonded form a cycloheteroalkyl or substituted cycloheteroalkyl ring;
each W is independently —NR⁸—;
each R⁸ is independently selected from hydrogen, alkyl, substituted alkyl, aryl and substituted aryl, or optionally, each R⁶ and R⁸ independently together with the atoms to which they are bonded form a cycloheteroalkyl or substituted cycloheteroalkyl ring;
p is an integer from one to five; and
R⁷ is selected from hydrogen, alkyl, substituted alkyl, acyl, substituted acyl, alkoxycarbonyl, substituted alkoxycarbonyl, aryl, substituted aryl, arylalkyl, substituted arylalkyl, and polyethylene glycol;
or a salt, hydrate or solvate thereof.

7. A compound according to claim 5, of formula (VII): 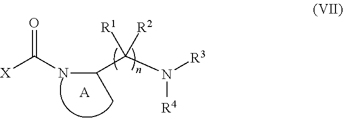
wherein:
X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;
the A ring is a heterocyclic 5 to 12-membered ring;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;
n is an integer from 1 to 10;
R³ is selected from alkyl, substituted alkyl, aryl, and substituted aryl;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or a salt, hydrate or solvate thereof.

8. A compound according to claim 5, of formula (VIII): 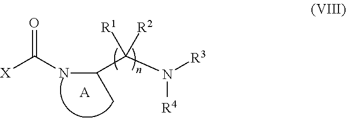
wherein:
X represents a residue of a ketone-containing opioid, wherein the hydrogen atom of the corresponding enolic group or reduced enolic group of the ketone is replaced by a covalent bond to —C(O)—N(A ring)-(C(R¹)(R²))ₙ—NR³R⁴; or wherein the hydrogen atom of an amino group that is generated from reductive amination of the ketone of the ketone-containing opioid is replaced by a covalent bond to —C(O)—N(A ring)-(C(R¹)(R²))ₙ—NR³R⁴;
the A ring is a heterocyclic 5 to 12-membered ring;
each R¹ is independently selected from hydrogen, alkyl, substituted alkyl, aryl, substituted aryl, acyl, and aminoacyl;
each R² is independently selected from hydrogen, alkyl, substituted alkyl, aryl, substituted aryl, acyl, and aminoacyl; or
R¹ and R² together with the carbon to which they are attached can form a cycloalkyl or substituted cycloalkyl group, or two R¹ or R² groups on adjacent carbon atoms, together with the carbon atoms to which they are attached, can form a cycloalkyl or substituted cycloalkyl group;
n is an integer from 1 to 10;
R³ is hydrogen;
R⁴ is selected from a residue of a D-amino acid; a residue of an N-acyl derivative of a D-amino acid; a residue of a polyethylene glycol derivative of a D-amino acid; a residue of L-proline; a residue of an N-acyl derivative of L-proline; a residue of a polyethylene glycol derivative of L-proline; a residue of a peptide composed of up to five amino acids wherein the amino acid of the peptide adjacent the nitrogen of —N(R³)(R⁴) is a residue of a D-amino acid; a residue of an N-acyl derivative of a peptide composed of up to five amino acids wherein the amino acid of the peptide adjacent the nitrogen of —N(R³)(R⁴) is a residue of a D-amino acid; and a residue of a polyethylene glycol derivative of a peptide composed of up to five amino acids wherein the amino acid of the peptide adjacent the nitrogen of —N(R³)(R⁴) is a residue of a D-amino acid;
or a salt, hydrate or solvate thereof.

9. (canceled)

10. A method of treating or preventing pain in a patient, which comprises administering an effective amount of a composition comprising the compound of claim 1.

11-13. (canceled)

14. A method of treating or preventing an unwanted side effect associated with use of an opioid agonist in a patient, which comprises administering an effective amount of a composition comprising the compound of claim 1.

15. (canceled)

16. A compound selected from the group consisting of the formulae: 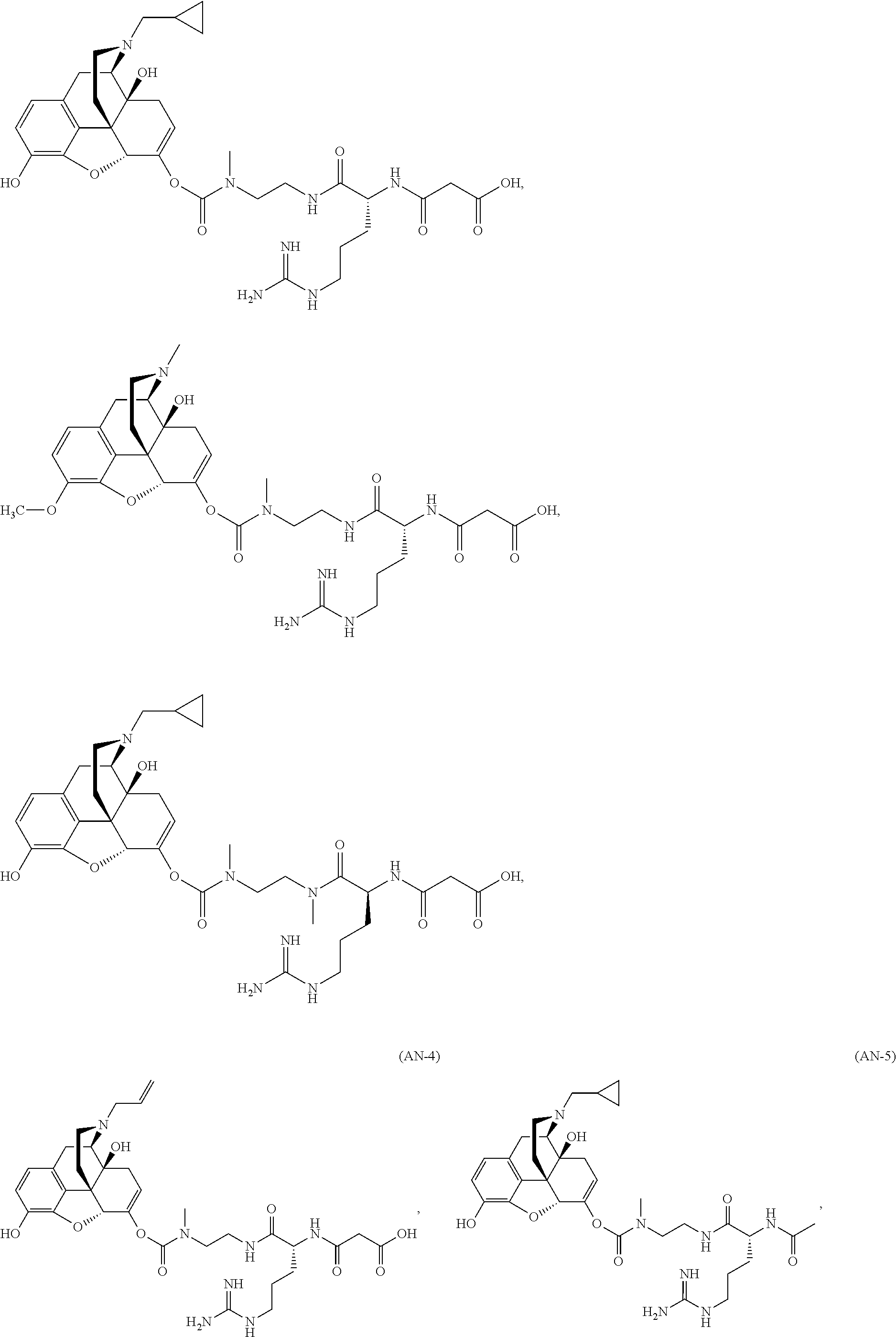 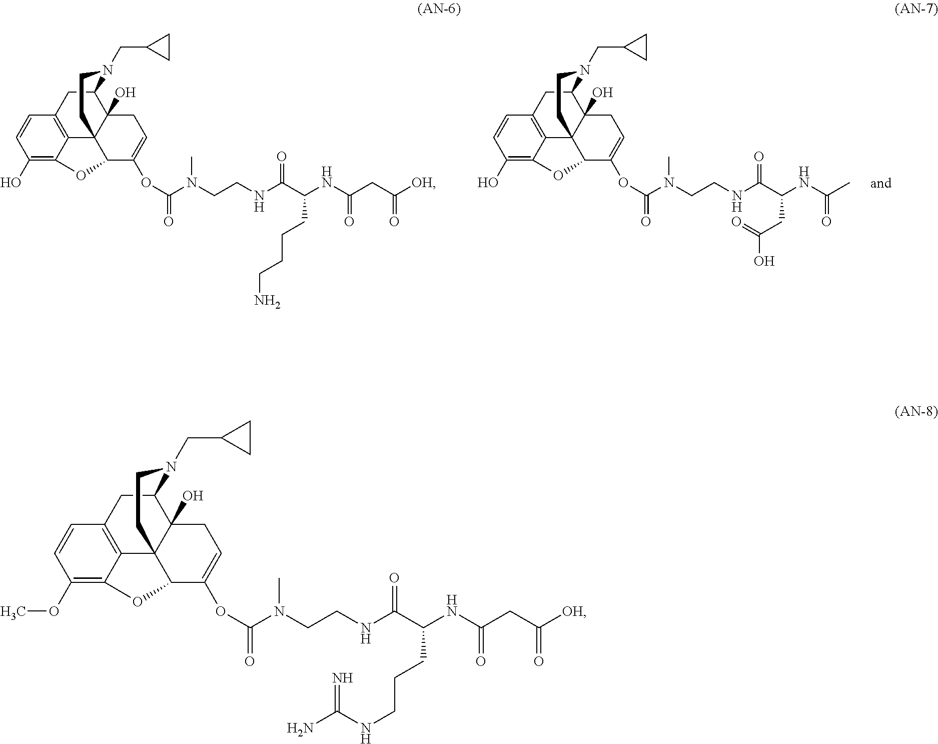
or salt or solvate or stereoisomer thereof.

17-23. (canceled)

24. A compound according to claim 1, of the formula: 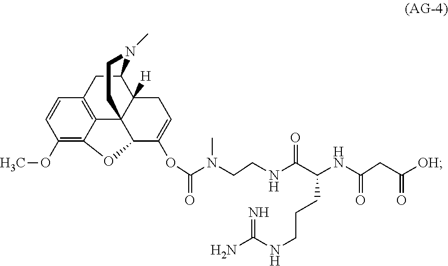
or salt or solvate or stereoisomer thereof.

25. A composition comprising a pharmaceutically acceptable carrier and a therapeutically effective amount of the compound of claim 1.

26. A composition comprising a pharmaceutically acceptable carrier and a therapeutically effective amount of the compound of claim 5.

27. A method of treating or preventing pain in a patient, which comprises administering an effective amount of a composition comprising the compound of claim 5.

28. A method of treating or preventing an unwanted side effect associated with use of an opioid agonist in a patient, which comprises administering an effective amount of a composition comprising the compound of claim 5.

29. A method of claim 28, wherein the unwanted side effect is selected from constipation, cough suppression, dry mouth, heartburn, myocardial depression, nausea, pruritus, urinary retention, vomiting, bloating, dry-mouth or heartburn.

30. A method of claim 14, wherein the unwanted side effect is selected from constipation, cough suppression, dry mouth, heartburn, myocardial depression, nausea, pruritus, urinary retention, vomiting, bloating, dry-mouth or heartburn.
